(19) United States
(12) Patent Application Publication
(10) Pub. No.: US 2026/0345805 A1
(43) Pub. Date: Nov. 5, 2026

(54) IMMUNOCONJUGATE COMPRISING IL-2 AND USE THEREOF

(71) Applicant: Beijing Chia Tai Innovative Pharmaceutical Co., Ltd., Beijing (CN)

(72) Inventors: Xiaojuan LIU, Beijing (CN); Daotian FU, Beijing (CN); Eric S.y. TSE, Beijing (CN)

(21) Appl. No.: 19/410,697

(22) Filed: Dec. 5, 2025

(30) Foreign Application Priority Data
Apr. 27, 2025 (CN) 202510542008.9

(51) Int. Cl. A61K 47/68; A61P 35/00; C07K 16/28

(52) U.S. Cl. CPC A61K 47/6813; A61K 47/6849; A61K 47/6889; A61P 35/00; C07K 16/2818

(57) ABSTRACT

The present disclosure pertains to the technical field of biopharmaceuticals and relates to an immunoconjugate with IL-2 and use thereof. The immunoconjugate includes laIL-2, and laIL-2, as compared to a wild-type IL-2 protein, includes at least one mutation at positions 38, 42, and/or 88. The present disclosure provides an immunoconjugate with laIL-2 and an anti-PD-1 antibody, which can target T cells in the tumor microenvironment, synergistically promote the activation of killer T cells, reverse exhausted T cells, significantly kill tumors, and simultaneously overcome PD-1 resistance, offering a novel therapy with clinical application prospects for tumor immunotherapy.

### CROSS-REFERENCE TO RELATED APPLICATIONS

This application claims priority under 35 U.S.C. § 119(b) to Chinese Application No. 202510542008.9, filed Apr. 27, 2025, the disclosure of which is incorporated herein by reference in its entirety.

### REFERENCE TO SEQUENCE LISTING SUBMITTED ELECTRONICALLY

This application contains a sequence listing, which is submitted electronically as a XML formatted sequence listing with a file name “689755_0033_Sequence Listing”, creation date of Dec. 4, 2025, and having a size of 40,075 bytes. The sequence listing submitted electronically is part of the specification and is herein incorporated by reference in its entirety.

### TECHNICAL FIELD

The present disclosure pertains to the technical field of biopharmaceuticals, and particularly relates to an immunoconjugate comprising IL-2 and use thereof.

### BACKGROUND

The tumor microenvironment (TME) generally limits the effects of immunotherapy by increasing Treg generation and/or reducing T-cell growth factors or their signaling. One major challenge is providing sufficient cytokines to reactivate CTLs or overcome Tregs.

Currently, PD-1/PD-L1 blocking antibodies have been used in first-line treatments for a variety of tumor indications, making them first-line standard therapy. However, only about 20% of patients respond to PD-1/PD-L1 blockade, while a vast majority of patients either fail to respond to the immune checkpoint therapy or rapidly develop resistance to this therapy.

In tumor immunotherapy, CD8⁺ T cells play a critical role in killing and controlling tumors, and interleukin-2 (IL-2) is a key T-cell growth factor that promotes the activation and proliferation of both Treg cells and T cells. IL-2 exerts its effects by binding to an IL-2 receptor, which mainly consists of three subunits: an IL-2 receptor α chain (IL-2Rα, also known as CD25), an IL-2 receptor β chain (IL-2Rβ, also known as CD122), and a common γ chain (IL-2Rγ, also known as CD132). The binding of IL-2 to IL-2Rα is mainly to mediate the binding of IL-2 to an IL-2 receptor, while the binding of IL-2 to IL-2Rβγ is mainly to transmit downstream biological activity signals. The expression of IL-2 receptors varies among naive T cells, activated T cells, exhausted T cells, and Treg cells, leading to differential effects of IL-2 on these effector cells. Tregs express all three subunits at high levels, enabling IL-2 to readily bind to Treg cells via high-affinity receptors, thereby enhancing the survival and proliferation of Tregs and promoting the immunosuppressive activity of Tregs.

Compared with Tregs, peripheral naive T cells and NK cells of tumors lack IL-2Rα expression and only express IL-2Rβγ receptors, and intratumoral activated or exhausted T cells exhibit upregulated IL-2R expression. In the current tumor immunotherapy, high-dose IL-2 used in clinical practice not only preferentially expands Treg cells and thereby inhibits tumor immunotherapy, but also activates peripheral T cells via binding to IL-2Rβγ on the surface of naive T cells.

There remains a need in the art for further improving IL-2 therapies.

### SUMMARY

The present disclosure provides an engineered low affinity IL-2 (laIL-2) to greatly reduce the affinity of IL-2 for IL-2Rα and IL-2Rβγ, so as to reduce the effect of IL-2 on Tregs and further reduce the potential toxicity of IL-2. Meanwhile, an immunoconjugate is also provided. laIL-2 is fused with an anti-PD-1 antibody, followed by expression, to form an immunoconjugate (anti-PD-1-laIL-2) so as to extend the half-life of IL-2 and simultaneously target laIL-2 to T cells in the tumor microenvironment, particularly CD8⁺ T cells. This design enables anti-PD-1 antibody and IL-2 to synergistically promote the activation of killer T cells in the tumor microenvironment through cis-interaction, reverse T cell exhaustion in the tumor microenvironment, and demonstrate significant tumor-killing effects, with the potential to overcome PD-1 resistance.

In order to achieve the objective described above, the present disclosure adopts the following technical solutions:

In a first aspect, the present disclosure provides an immunoconjugate, and the immunoconjugate comprises laIL-2, wherein laIL-2, as compared to wild-type IL-2, comprises at least one mutation selected from the group consisting of mutations at positions 38, 42, and 88 of an amino acid sequence; an amino acid sequence of the wild-type IL-2 is the amino acid sequence set forth in SEQ ID NO: 1.

In a second aspect, the present disclosure also provides a nucleic acid encoding the aforementioned immunoconjugate.

In a third aspect, the present disclosure also provides a vector comprising the aforementioned nucleic acid.

In a fourth aspect, the present disclosure also provides a cell comprising the aforementioned nucleic acid or the aforementioned vector.

In a fifth aspect, the present disclosure also provides a pharmaceutical composition comprising the aforementioned immunoconjugate, the aforementioned nucleic acid, the aforementioned vector, and/or the aforementioned cell.

In a sixth aspect, the present disclosure provides use of the aforementioned immunoconjugate, the aforementioned nucleic acid, the aforementioned vector, the aforementioned cell, or the aforementioned pharmaceutical composition for preparing a medicament for preventing and/or treating cancer.

One or more technical solutions in the embodiments of the present disclosure have at least the following technical effects or advantages:

The immunoconjugate (anti-PD-1-laIL-2) formed by fusing laIL-2 with an anti-PD-1 antibody can target T cells in the tumor microenvironment. While blocking the PD-1-PD-L1 binding, it can synergistically promote the activation of killer T cells, reverse T cell exhaustion, and significantly kill tumors. The immunoconjugate can achieve 100% tumor regression in the MC38 cell tumor model mice with no significant weight loss being observed, demonstrating markedly superior safety. Meanwhile, laIL-2 reduces binding to both IL-2Rα and IL-2Rβγ receptors, thereby reducing binding to and activation of non-target cells and lowering peripheral toxicity. In addition, the immunoconjugate prolongs the half-life of IL-2, simplifies the treatment process, has the potential to overcome PD-1 resistance, and offers a novel scheme for tumor immunotherapy, with prominent druggability advantages.

### BRIEF DESCRIPTION OF THE DRAWINGS

FIG. 1 is a schematic diagram of the molecular structures of an immunoconjugate comprising IL-2.

FIG. 2 shows the results for the binding of immunoconjugates to a human PD-1 protein detected by ELISA.

FIG. 3 shows the results for the binding of immunoconjugates to IL-2Rα detected by ELISA.

FIG. 4 shows the results for the binding of immunoconjugates to IL-2Rβγ detected by ELISA.

FIG. 5 shows the results for the binding of IL-2 in immunoconjugates to a receptor, as detected by using HEK-blue-IL-2 cells.

FIG. 6 shows the results for the affinity of immunoconjugates for human PD-1, as detected by using HEK-blue-IL-2/hPD-1 cells.

FIG. 7 shows the results for the activity of IL-2 in immunoconjugates, as detected by using HEK-blue-IL-2 cells.

FIG. 8 shows the results for the activity of IL-2 in immunoconjugates, as detected by using HEK-blue-IL-2/PD-1 cells.

FIG. 9 shows the results for the activation of downstream signals by immunoconjugates comprising IL-2, as detected by using Naïve PBMC cells.

FIG. 10 shows the results for the cell death and cell proliferation within 1-7 days of the co-incubation of immunoconjugates comprising IL-2 and a receptor, as detected by using Naïve PBMC cells.

FIG. 11 shows the results for the activation of downstream signals by immunoconjugates comprising IL-2, as detected by using CD3/CD28-activated PBMC cells.

FIG. 12 shows the results for the activation of downstream signals by immunoconjugates comprising IL-2, as detected by using CD3/CD28-activated PBMC cells.

FIG. 13 shows the in vivo detection results for the inhibitory effects of candidate molecules on tumor growth in the MC38 cell tumor model mice.

FIG. 14 shows the in vivo detection results for the effects of candidate molecules on the weight of tumor-bearing mice in the MC38 cell tumor model mice.

FIG. 15 shows the in vivo detection results for the effects of candidate molecules on the mortality of tumor-bearing mice in the MC38 cell tumor model mice.

FIG. 16 shows the effect of candidate molecules on the body weight of tumor-bearing mice in the pancreatic cancer model.

### DETAILED DESCRIPTION

### I. Definitions

In order to make the objectives, technical solutions, and advantages of the embodiments of the present disclosure clearer, the technical solutions of the embodiments of the present disclosure will be clearly and completely described below with reference to the drawings related to the embodiments of the present disclosure. Apparently, the described embodiments are just a part, but not all of the embodiments of the present disclosure. Based on the described embodiments of the present disclosure, all other embodiments obtained by those of ordinary skills in the art without any inventive work shall fall within the protection scope of the present disclosure.

The present disclosure may be implemented in other specific forms without departing from the basic attributes of the present disclosure. It should be understood that, in the absence of conflicts, any and all embodiments of the present disclosure may be combined with the technical features in any other embodiment or multiple other embodiments to obtain additional embodiments. The present disclosure includes additional embodiments resulting from such combinations.

All publications and patents mentioned in the present disclosure are incorporated herein by reference in their entirety. If uses or terms used in any of the publications and patents incorporated by reference conflict with uses or terms used in the present disclosure, the uses and terms of the present disclosure prevail.

The section headings used herein are for organizational purposes only and are not to be construed as limiting the subject matter described.

Unless otherwise defined, all technical and scientific terms used in the present disclosure have the same meaning as commonly used in the art to which the present disclosure pertains. For purposes of interpreting this specification, the following definitions will apply, and whenever appropriate, terms used in the singular shall also include the plural and vice versa.

Unless the context clearly dictates otherwise, the expressions “a” and “an” as used herein include plural referents. For example, reference to “a cell” includes a plurality of such cells and equivalents known to those skilled in the art, and so forth.

As used herein, the term “about” or “approximately” refers to a value similar to the reference value, and indicates a range of ±20% of the value following it. In some embodiments, the term “about” indicates a range of ±10% of the value following it. In some embodiments, the term “about” indicates a range of ±5% of the value following it. In certain embodiments, unless otherwise stated or otherwise evident from the context, the term “approximately” or “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value (unless such number would exceed 100% of a possible value).

The numerical ranges as used herein should be construed as having listed all numbers within that range. For example, a range of 1 to 20 should be understood to include any number, combination of numbers, or sub-range from the group consisting of 1, 2, 3, 4, 5, 6, 7, 8, 9, 10, 11, 12, 13, 14, 15, 16, 17, 18, 19, or 20 As used herein, the term “comprises” or “comprising” refers to “including but not limited to”. The term is intended to be open-ended, to specify the presence of any stated features, elements, integers, steps, or components, but does not preclude the presence or addition of one or more other features, elements, integers, steps, components, or groups thereof.

As used herein, the term “optional”, “any”, “arbitrary”, or “any one” means that the event or circumstance subsequently described may, but not necessarily, occur, and that the description includes instances where the event or circumstance occurs or does not occur. As used in the present disclosure, “an” and “a” are used in the present disclosure to refer to one or more grammatical objects.

As used herein, “and/or” should be understood to refer to any one of the options or a combination of any two or more of the options.

As used herein, the term “nucleic acid” includes RNA, DNA, and cDNA molecules. It should be understood that due to the degeneracy of the genetic code, a large number of nucleotide sequences encoding a given protein can be generated. “Nucleic acid sequence”, “nucleotide sequence”, or “polynucleotide sequence” are used interchangeably and refer to a contiguous nucleic acid sequence. The sequence may be single-stranded or double-stranded DNA or RNA, e.g., mRNA.

As used herein, the terms “peptide”, “polypeptide”, and “protein” are used interchangeably herein and refer to polymeric forms of amino acids of any length, and these polymeric forms may include coded and non-coded amino acids, chemically or biochemically modified or derivatized amino acids, and polypeptides having modified peptide skeletons. The starting point of a protein or polypeptide is referred to as the “N-terminus” (or amino-terminus, NH₂-terminus, or amine-terminus), which refers to the free amine (—NH₂) group of the first amino acid residue of a protein or polypeptide. The terminus of a protein or peptide is referred to as “C-terminus” (or carboxyl-terminus, or COOH-terminus), which refers to the free carboxyl (—COOH) of the last amino acid residue of a protein or peptide.

As used herein, the term “identity” refers to the degree to which sequences are identical on a nucleotide-by-nucleotide or amino acid-by-amino acid basis in a comparison window. The “percent sequence identity” can be calculated by the following steps: comparing two optimally aligned sequences in a comparison window; determining the number of positions at which identical nucleic acid bases (e.g., A, T, C, G, and I) or identical amino acid residues (e.g., Ala, Pro, Ser, Thr, Gly, Val, Leu, Ile, Phe, Tyr, Trp, Lys, Arg, His, Asp, Glu, Asn, Gln, Cys, and Met) occur in both sequences to obtain the number of matched positions; dividing the number of matched positions by the total number of positions in the comparison window (i.e., the window size); and multiplying the result by 100 to yield the percent sequence identity. Optimal alignment for determining the percent sequence identity can be achieved in a variety of ways known in the art, for example, using publicly available computer software such as BLAST, BLAST-2, ALIGN, or Megalign (DNASTAR) software. Those skilled in the art can determine suitable parameters for alignment of sequences, including any algorithms required to achieve maximum alignment within the full-length sequence range or target sequence region being compared. Herein, with respect to antibody sequences, the percent amino acid sequence identity is determined by optimally aligning a candidate antibody sequence to a given antibody sequence, and in a preferred embodiment, the optimal alignment is performed according to the Kabat numbering scheme. Herein, without specifying the comparison window (i.e., the target antibody region to be compared), it will be applicable to align over the full length of the given antibody sequence.

In the description herein, reference to “some embodiments” describes a subset of all possible embodiments, but it can be understood that “some embodiments” may be the same subset or different subsets of all possible embodiments, and can be combined with one another without conflict.

As used herein, the term “antibody” is used in its broadest sense and specifically encompasses monoclonal antibodies (including full-length monoclonal antibodies), polyclonal antibodies, multispecific antibodies (e.g., bispecific antibodies), and antibody fragments or synthetic polypeptides carrying one or more CDRs or derived from CDR sequences, so long as these polypeptides exhibit the desired biological activity. Antibodies (Abs) and immunoglobulins (Igs) are glycoproteins having the same structural characteristics. “Antibody” may also refer to an immunoglobulin and an immunoglobulin fragment, whether naturally occurring or partially or completely synthetically (e.g., recombinantly) produced, including any fragment thereof that comprises at least a portion of the variable region of the immunoglobulin molecule that retains the binding specificity ability of the full-length immunoglobulin. Thus, antibodies include any protein having a binding domain that is homologous or substantially homologous to an immunoglobulin antigen-binding domain. Antibodies include antibody fragments, such as anti-tumor stem cell antibody fragments. As used herein, the term antibody thus includes synthetic antibodies, recombinantly produced antibodies, multispecific antibodies (e.g., bispecific antibodies), human antibodies, non-human antibodies, humanized antibodies, chimeric antibodies, intracellular antibodies, and antibody fragments, such as, but not limited to, Fab fragments, Fab′ fragments, F(ab′)₂ fragments, Fv fragments, disulfide-stabilized Fvs (dsFvs), Fd fragments, Fd′ fragments, single-chain Fvs (scFvs), single-chain Fabs (scFabs), diabodies, anti-idiotypic (anti-Id) antibodies, or antigen-binding fragments of any of the antibodies described above. The antibodies provided herein include members of any immunoglobulin type (e.g., IgG, IgM, IgD, IgE, IgA, and IgY), any class (e.g., IgG1, IgG2, IgG4, IgA1, and IgA2), or any subclass (e.g., IgG2a and IgG2b) (“type” and “class”, as well as “subtype” and “subclass”, are used interchangeably herein).

“Polypeptide chain” is a chain structure formed by linking amino acids via peptide bonds. An antibody molecule generally consists of four polypeptide chains, including two identical heavy chains (H chains) and two identical light chains (L chains), which are linked together by disulfide bonds to form a Y-shaped structure. Each heavy chain consists of one variable region (VH) and three constant regions (CH1, CH2, and CH3), while each light chain consists of one variable region (VL) and one constant region (CL).

As used herein, the term “Fc”, “Fc domain”, or “Fc region” is used to define the C-terminal region of an antibody heavy chain that comprises at least a portion of the constant region. The term includes natural sequence Fc regions and variant Fc regions. The IgG Fc region comprises IgG CH2 and IgG CH3 domains. An Fc molecule consists of monomeric polypeptides that can be linked by covalent (i.e., disulfide bonds) and non-covalent associations into dimeric or multimeric forms. The number of intermolecular disulfide bonds between monomeric subunits of natural Fc molecules ranges from 1 to 4, depending on the class (e.g., IgG, IgA, and IgE) or subclass (e.g., IgG1, IgG2, IgG3, IgA1, and IgA2).

As used herein, the term “Knob-into-hole (KIH)” refers to a technique for achieving specific dimerization of two heterologous polypeptide chains through spatial complementarity by engineering the Fc region of an antibody. In the Knob chain, amino acid mutations that increase steric bulk (e.g., Thr366Trp and T366W) and site mutations that stabilize interactions (e.g., S354C) are introduced into the Fc region, forming a protrusion structure. In the Hole chain, amino acid mutations that reduce steric bulk (e.g., T366S and Y407V) and complementary mutations (e.g., L368A and Y349C) are introduced into the corresponding region of the Fc region, forming a cavity structure. The protrusion structure of the Knob chain and the cavity structure of the Hole chain achieve specific binding through spatial matching, driving heterodimerization (i.e., binding between the Knob chain and the Hole chain) while significantly suppressing the formation of homodimers (Knob-Knob or Hole-Hole).

As used herein, the term “hinge” or “hinge region” refers to a flexible polypeptide comprising the amino acids between the first and second constant domains of an antibody.

As used herein, the terms “full-length antibody”, “intact antibody”, and “whole antibody” are used interchangeably herein and refer to an antibody having a structure substantially similar to the structure of a natural antibody. A full-length antibody comprises two full-length antibody light chains each comprising a light chain variable domain and a light chain constant domain, and two full-length antibody heavy chains each comprising a heavy chain variable domain, a first constant domain, a hinge region, a second constant domain, and a third constant domain.

As used herein, the term “LALA mutation” refers to the substitution of leucine (Leu) with alanine (Ala) at positions 234 and 235 of the Fc region of an antibody, i.e., L234A and L235A. Such mutations can reduce the binding of the antibody to an Fcγ receptor (FcγR) and complement component C1q, thereby reducing the effector functions of the antibody, such as antibody-dependent cell-mediated cytotoxicity (ADCC) and complement-dependent cytotoxicity (CDC). The “LALAPG mutation” is based on the LALA mutation by further replacing the proline (Pro) with glycine (Gly) at position 329, i.e., P329G. LALAPG mutants are able to inhibit binding to FcγR and Clq more significantly, while maintaining binding to FcRn and stability of the Fc unaffected. This makes LALAPG mutants more effective in reducing the effector function of the antibody.

As used herein, the term “antibody-dependent cell-mediated cytotoxicity (ADCC)” refers to the effect of NK cells, macrophages, neutrophils, etc., expressing IgG Fc receptors, killing target cells such as virus-infected cells and tumor cells by binding to the Fc fragment of an IgG antibody that has bound to the surface of these target cells.

As used herein, the term “immunoconjugate” refers to a conjugate comprising an IL-2 (wild-type or variant thereof) molecule and an antibody. By directly targeting IL-2 molecules, for example, to the tumor microenvironment, such immunoconjugates significantly enhance the efficacy of IL-2 therapy. In accordance with the disclosure herein, the antibody contained in the immunoconjugate may be a whole antibody or immunoglobulin, or a portion or variant thereof having a biological function, such as antigen-specific binding affinity.

As used herein, the term “laIL-2” is “low affinity IL-2”.

As used herein, the term “affinity” or “binding affinity” refers to the strength of the sum of non-covalent interactions between a single binding site of a molecule and its binding ligand (e.g., an antigen). The binding affinity can generally be represented by the dissociation constant (KD). The dissociation constant (KD) is the ratio of the dissociation rate (kd) to the association rate (ka), i.e., KD=kd/ka.

Affinity can be measured by conventional methods known in the art, such as surface plasmon resonance (SPR).

As used herein, the term “replacement” or “amino acid replacement” refers to the replacement of an amino acid residue in a sequence.

Also provided are “conservative sequence modifications” of the sequences described in the sequence listing described herein, i.e., nucleotide and amino acid sequence modifications that do not eliminate the binding of an antibody encoded by a nucleotide sequence or containing an amino acid sequence to an antigen. These conservative sequence modifications include conservative nucleotide and amino acid replacement, and nucleotide and amino acid additions and deletions. For example, modifications can be introduced into the sequences described herein by standard techniques known in the art (e.g., site-directed mutagenesis and PCR-mediated mutagenesis). Conservative sequence modifications include conservative amino acid substitutions in which an amino acid residue is replaced with an amino acid residue with a similar side chain. Families of amino acid residues with similar side chains have been defined in the art. These families include amino acids with basic side chains (e.g., lysine, arginine, and histidine), amino acids with acidic side chains (e.g., aspartic acid and glutamic acid), amino acids with uncharged polar side chains (e.g., glycine, asparagine, glutamine, serine, threonine, tyrosine, cysteine, and tryptophan), amino acids with non-polar side chains (e.g., alanine, valine, leucine, isoleucine, proline, phenylalanine, and methionine), amino acids with β-branched side chains (e.g., threonine, valine, and isoleucine), and amino acids with aromatic side chains (e.g., tyrosine, phenylalanine, tryptophan, and histidine). Methods for identifying nucleotide and amino acid conservative substitutions that do not eliminate antigen binding are well known in the art (e.g., see Brummell et al., Biochem. 32:1180-1187 (1993); Kobayashi et al., Protein Eng. 12(10):879-884 (1999); and Burks et al., Proc. Natl. Acad. Sci. USA 94:412-417 (1997)).

As used herein, “mutated amino acid” includes “replacement, repetition, deletion, or addition of one or more amino acids”. In the present disclosure, the term “mutation” refers to a change in an amino acid sequence. In a specific embodiment, the term “mutation” refers to “replacement”.

As used herein, the term “pharmaceutically acceptable carrier” is intended to include any and all solvents, dispersion media, coatings, antibacterial and antifungal agents, isotonizing agents, absorption delaying agents, and the like that are compatible with pharmaceutical administration. Except insofar as any conventional media or reagents are incompatible with the active compound, use of the conventional media or reagents in these compositions is contemplated. The pharmaceutically acceptable carrier includes diluents, fillers, salts, buffering agents, stabilizers, solubilizers, and other materials well known in the art.

As used herein, the term “pharmaceutical composition” may also be administered to a patient or subject in need of such treatment in any suitable route of administration, such as oral, intravenous, parenteral, rectal, transpulmonary, or topical administration. For oral administration, the pharmaceutical composition may be formulated into an oral formulation, for example, an oral solid formulation, such as a tablet, a capsule, a pill, and a granule; or an oral liquid formulation, such as an oral solution, an oral suspension, and a syrup. When formulated into an oral formulation, the pharmaceutical formulation may further comprise a suitable filler, binder, disintegrant, lubricant, and the like. The pharmaceutical composition of the present disclosure may be formulated into any pharmaceutically acceptable dosage form for oral, nasal, topical (including buccal and sublingual), rectal, vaginal, and/or parenteral administration. For example, it may be formulated into a tablet, a pastille, a capsule, a pill, a solution, a suspension, a syrup, an injection, a suppository, an inhalant, or a spray.

As used herein, the term “inhibiting” is used interchangeably with “reducing”, “silencing”, “down-regulating”, and other similar terms, and includes any level of inhibition.

As used herein, the term “treat”, “treating”, or “treatment” includes inhibiting, slowing down, stopping, or reversing the progression or severity of an existing symptom or condition. Thus, treatment includes prevention, therapy, and/or cure. In addition, “treatment”, “treating”, “alleviating”, or “ameliorating” can be used interchangeably herein. Prevention refers to the prevention of the potential disease and/or the prevention of the worsening of symptoms or disease progression. “Ameliorating” refers to alleviating, slowing down, stopping, or reversing at least one indicator of the severity of a condition or disease. The severity of the indicator can be determined by subjective or objective measures known to those skilled in the art. As used herein, “therapeutic effect” refers to an effect resulting from the treatment of an individual that alters, generally mitigates, or ameliorates symptoms of a disease or disease condition, or cures a disease or disease condition.

As used herein, the term “therapeutic agent” refers to an agent that has a therapeutic, diagnostic, and/or prophylactic effect and/or elicits a desired biological and/or pharmacological effect when administered to a subject.

As used herein, the term “therapeutically effective amount” or “therapeutically effective dose” refers to an amount of a substance, a compound, a material, or a composition comprising a compound that is at least sufficient to produce a therapeutic effect after administration to a subject. Thus, it is the amount necessary to prevent, cure, ameliorate, arrest, or partially arrest the symptoms of a disease or disorder. As used herein, “prophylactically effective amount” or “prophylactically effective dose” refers to an amount of a substance, a compound, a material, or a composition comprising a compound that will have the desired prophylactic effect when administered to a subject, e.g., preventing or delaying the occurrence or recurrence of a disease or a symptom, or reducing the possibility of the occurrence or recurrence of a disease or a symptom. A complete prophylactically effective dose need not occur by administration of one dose, and may occur only after administration of a series of doses. Thus, a prophylactically effective amount may be administered in one or more administrations.

As used herein, the term “patient” refers to any animal, such as a mammal or a marsupial. Subject matter of the present disclosure includes, but is not limited to, humans, non-human primates (e.g., rhesus monkeys or other types of macaques), mice, pigs, horses, donkeys, cattle, sheep, rats, and any species of poultry.

As used herein, “preventing” and “prevention” are used interchangeably. These terms refer to an approach for obtaining beneficial or desired results, including but not limited to prophylactic benefits. To obtain a prophylactic benefit, a conjugate or composition can be administered to a patient at risk of developing a particular disease, or to a patient who reports one or more pathological symptoms of a disease, even though a diagnosis of the disease may not have been made.

As used herein, the term “cancer” or “tumor” or “hyperproliferative disease” refers to the presence of cells having typical characteristics of oncogenic cells (e.g., uncontrolled proliferation, immortality, metastatic potential, rapid growth and proliferation rate, and certain characteristic morphological features). The cancer cells are generally in the form of a tumor, but these cells may be present alone in the animal, or may be non-tumorigenic cancer cells, such as leukemia cells. The term “cancer” includes premalignant cancers and malignant cancers. The term “premalignant lesion” described herein refers to a lesion that is not cancerous but has the potential to become cancerous. It also includes the term “premalignant disorder” or “potential malignant disorder”. In particular, it denotes benign morphologically and/or histologically altered tissues, which carries an above-normal risk of malignant transformation, as well as diseases or patient habits that do not necessarily alter the clinical presentation of local tissues but are associated with an above-normal risk of precancerous lesions or cancer development in the tissues (leukoplakia, erythroplakia, erythroleukoplakia, lichen planus (lichenoid reaction), and any lesions or areas showing cellular abnormalities or dysplasia on histological examination).

Cancers include, but are not limited to, B-cell cancers (e.g., multiple myeloma), Waldenstrom macroglobulinemia, heavy chain diseases (e.g., α chain disease, γ chain disease, and μ chain disease), benign monoclonal gammopathy, as well as immunocytic amyloidosis, melanoma, breast cancer, lung cancer, bronchial cancer, colorectal cancer, prostate cancer, pancreatic cancer, gastric cancer, ovarian cancer, bladder cancer, cancer of the brain or central nervous system, cancer of the peripheral nervous system, esophageal cancer, cervical cancer, uterine or endometrial cancer, cancer of the oral cavity or pharynx, liver cancer, kidney cancer, testicular cancer, biliary tract cancer, cancer of the small intestine or appendix, salivary gland cancer, thyroid cancer, adrenal cancer, osteosarcoma, chondrosarcoma, cancer of blood tissues, and the like. Other non-limiting examples of cancer types applicable to the methods encompassed by the present disclosure include human sarcomas and cancers, such as fibrosarcoma, myxosarcoma, liposarcoma, chondrosarcoma, osteogenic sarcoma, chordoma, angiosarcoma, endotheliosarcoma, lymphangiosarcoma, lymphangioendothelioma, synovioma, mesothelioma, Ewing's tumor, leiomyosarcoma, rhabdomyosarcoma, colon cancer, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, cholangiocarcinoma, liver cancer, choriocarcinoma, seminoma, embryonal carcinoma, Wilms tumor, cervical cancer, bone cancer, brain tumors, testicular cancer, lung cancer, small cell lung cancer, bladder cancer, epithelial cancer, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, such as acute lymphocytic leukemia and acute myelocytic leukemias (myeloblastic, promyelocytic, myelomonocytic, and monocytic leukemias, as well as erythroleukemia); chronic leukemias (chronic myelocytic (granulocytic) leukemia and chronic lymphocytic leukemia); and polycythemia vera, lymphoma (Hodgkin and non-Hodgkin disease), multiple myeloma, Waldenstrom macroglobulinemia, and heavy chain diseases. In some embodiments, the cancer is liver cancer, bladder cancer, melanoma, or colon cancer.

### II. Detailed Description of Specific Embodiments

In a first aspect, the present disclosure provides an immunoconjugate, and the immunoconjugate comprises laIL-2, wherein laIL-2, as compared to wild-type IL-2, comprises at least one mutation selected from the group consisting of mutations at positions 38, 42, and 88 of an amino acid sequence; an amino acid sequence of the wild-type IL-2 is the amino acid sequence set forth in SEQ ID NO: 1.

In some embodiments, the mutation at position 38 of the amino acid sequence is R38L, R38D, R38E, or R38W.

In some preferred embodiments, the mutation at position 38 of the amino acid sequence is R38L.

In some embodiments, the mutation at position 42 of the amino acid sequence is F42A, F42Q, or F42E.

In some preferred embodiments, the mutation at position 42 of the amino acid sequence is F42A.

In some embodiments, the mutation at position 88 of the amino acid sequence is N88D, N88E, N88Q, N88H, N88R, N88Y, or N88W.

In some preferred embodiments, the mutation at position 88 of the amino acid sequence is N88D.

In some embodiments, laIL-2, as compared to a wild-type IL-2 protein, comprises amino acid mutations:
(1) R38L and F42A; or
(2) R38L, F42A, and N88D.

In some preferred embodiments, the amino acid sequence further comprises a mutation at position 3, and the mutation at position 3 is T3A.

In some preferred embodiments, the amino acid sequence further comprises a mutation at position 125, and the mutation at position 125 is C125A or C125S.

In some embodiments, laIL-2 comprises an amino acid sequence selected from an amino acid sequence set forth in any one of SEQ ID NOs: 2-5, or an amino acid sequence having at least 90%, 91%, 92%, 93%, 94%, 95%, 96%, 97%, 98%, or 99% identity thereto.

In some preferred embodiments, laIL-2 comprises an amino acid sequence set forth in SEQ ID NO: 3 or 5, or an amino acid sequence having at least 90%, 91%, 92%, 93%, 94%, 95%, 96%, 97%, 98%, or 99% identity thereto.

In some embodiments, the immunoconjugate comprises: an antibody specifically binding to programmed death receptor 1 (an anti-PD-1 antibody) or an antigen-binding fragment thereof, a linker, and laIL-2.

In some preferred embodiments, the antibody or the antigen-binding fragment thereof is selected from the group consisting of a single-chain Fv (scFv) fragment, a Fab fragment, a Fab′ fragment, a F(ab′) 2, an Fv fragment, a disulfide-stabilized Fv (dsFv) fragment, a disulfide-stabilized scFv (dsscFv), a (dsFv) 2, and a VHH.

In some preferred embodiments, the antibody or the antigen-binding fragment thereof is a Fab fragment.

In some preferred embodiments, the immunoconjugate further comprises an Fc.

In some preferred embodiments, the immunoconjugate comprises: a Fab fragment of an anti-PD-1 antibody, an Fc, a linker, and laIL-2.

In some embodiments, the anti-PD-1 antibody comprises:
an LCDR1 of the amino acid sequence set forth in SEQ ID NO: 29, an LCDR2 of the amino acid sequence set forth in SEQ ID NO: 30, an LCDR3 of the amino acid sequence set forth in SEQ ID NO: 31, an HCDR1 of the amino acid sequence set forth in SEQ ID NO: 32, an HCDR2 of the amino acid sequence set forth in SEQ ID NO: 33, and an HCDR3 of the amino acid sequence set forth in SEQ ID NO: 34.

In some embodiments, the anti-PD-1 antibody comprises:
a light chain variable region comprising the amino acid sequence set forth in SEQ ID NO: 35 or an amino acid sequence having at least 80% sequence identity to SEQ ID NO: 35; and a heavy chain variable region comprising the amino acid sequence set forth in SEQ ID NO: 36 or an amino acid sequence having at least 80% sequence identity to SEQ ID NO: 36.

In some embodiments, the Fc is derived from IgG1, IgG2, IgG3, or IgG4.

In some embodiments, the Fc is derived from IgG1.

In some embodiments, the immunoconjugate comprises:
(1) a first polypeptide chain comprising, from N-terminus to C-terminus, laIL-2, a linker, and an Fc in sequence, wherein laIL-2 is linked to the N-terminus of the Fc via the linker; a second polypeptide chain comprising a heavy chain Fd fragment of the anti-PD-1 antibody and an Fc; a third polypeptide chain comprising one light chain of the anti-PD-1 antibody; or
(2) a first polypeptide chain comprising, from N-terminus to C-terminus, a heavy chain Fd fragment of the anti-PD-1 antibody, an Fc, a linker, and laIL-2 in sequence, wherein laIL-2 is linked to the C-terminus of the Fc via the linker; a second polypeptide chain comprising a heavy chain Fd fragment of the anti-PD-1 antibody and an Fc; two third polypeptide chains each comprising one light chain of the anti-PD-1 antibody.

In some embodiments, one polypeptide chain of the Fc comprises T366W and S354C mutations, and the other polypeptide chain comprises T366S, L368A, Y407V, and Y349C mutations; the Fc optionally further comprises an LALA mutation or an LALAPG mutation.

In some embodiments, the linker is 1G4S, 2G4S, or 3G4S.

In some preferred embodiments, the linker is 1G4S.

In some embodiments, the Fc comprises an amino acid sequence selected from the group consisting of amino acid sequences set forth in SEQ ID NOs: 9-12.

In some embodiments, the heavy chain Fd fragment of the anti-PD-1 antibody comprises the amino acid sequence set forth in SEQ ID NO: 14.

In some embodiments, the light chain of the anti-PD-1 antibody comprises the amino acid sequence set forth in SEQ ID NO: 13.

In some embodiments, the immunoconjugate comprises:
(1) a first polypeptide chain comprising, from N-terminus to C-terminus, laIL-2, 1G4S, and an Fc in sequence, wherein laIL-2 is linked to the N-terminus of the Fc via 1G4S, and amino acid sequences of laIL-2 and the Fc are amino acid sequences set forth in SEQ ID NOs: 2 and 9, respectively; a second polypeptide chain comprising a heavy chain Fd fragment of the anti-PD-1 antibody and an Fc, wherein amino acid sequences of the heavy chain Fd fragment and the Fc are amino acid sequences set forth in SEQ ID NOs: 14 and 10, respectively; a third polypeptide chain comprising one light chain of the anti-PD-1 antibody, wherein an amino acid sequence of the light chain is the amino acid sequence set forth in SEQ ID NO: 13;
(2) a first polypeptide chain comprising, from N-terminus to C-terminus, a heavy chain Fd fragment of the anti-PD-1 antibody, an Fc, 1G4S, and laIL-2 in sequence, wherein laIL-2 is linked to the C-terminus of the Fc via 1G4S, and amino acid sequences of the heavy chain Fd fragment, the Fc, and laIL-2 are amino acid sequences set forth in SEQ ID NOs: 14, 11, and 4, respectively; a second polypeptide chain comprising a heavy chain Fd fragment of the anti-PD-1 antibody and an Fc, wherein amino acid sequences of the heavy chain Fd fragment and the Fc are amino acid sequences set forth in SEQ ID NOs: 14 and 12, respectively; two third polypeptide chains each comprising one light chain of the anti-PD-1 antibody, wherein an amino acid sequence of the light chain is the amino acid sequence set forth in SEQ ID NO: 13;
(3) a first polypeptide chain comprising, from N-terminus to C-terminus, a heavy chain Fd fragment of the anti-PD-1 antibody, an Fc, 1G4S, and laIL-2 in sequence, wherein laIL-2 is linked to the C-terminus of the Fc via 1G4S, and amino acid sequences of the heavy chain Fd fragment, the Fc, and laIL-2 are amino acid sequences set forth in SEQ ID NOs: 14, 11, and 3, respectively; a second polypeptide chain comprising a heavy chain Fd fragment of the anti-PD-1 antibody and an Fc, wherein amino acid sequences of the heavy chain Fd fragment and the Fc are amino acid sequences set forth in SEQ ID NOs: 14 and 12, respectively; two third polypeptide chains each comprising one light chain of the anti-PD-1 antibody, wherein an amino acid sequence of the light chain is the amino acid sequence set forth in SEQ ID NO: 13; or
(4) a first polypeptide chain comprising, from N-terminus to C-terminus, laIL-2, 1G4S, and an Fc in sequence, wherein laIL-2 is linked to the N-terminus of the Fc via 1G4S, and amino acid sequences of laIL-2 and the Fc are amino acid sequences set forth in SEQ ID NOs: 5 and 9, respectively; a second polypeptide chain comprising a heavy chain Fd fragment of the anti-PD-1 antibody and an Fc, wherein amino acid sequences of the heavy chain Fd fragment and the Fc are amino acid sequences set forth in SEQ ID NOs: 14 and 10, respectively; a third polypeptide chain comprising one light chain of the anti-PD-1 antibody, wherein an amino acid sequence of the light chain is the amino acid sequence set forth in SEQ ID NO: 13.

In some preferred embodiments, the immunoconjugate comprises:
(1) a first polypeptide chain comprising, from N-terminus to C-terminus, a heavy chain Fd fragment of the anti-PD-1 antibody, an Fc, 1G4S, and laIL-2 in sequence, wherein laIL-2 is linked to the C-terminus of the Fc via 1G4S, and amino acid sequences of the heavy chain Fd fragment, the Fc, and laIL-2 are amino acid sequences set forth in SEQ ID NOs: 14, 11, and 3, respectively; a second polypeptide chain comprising a heavy chain Fd fragment of the anti-PD-1 antibody and an Fc chain, wherein amino acid sequences of the heavy chain Fd fragment and the Fc are amino acid sequences set forth in SEQ ID NOs: 14 and 12, respectively; a third polypeptide chain comprising a light chain of the anti-PD-1 antibody, wherein an amino acid sequence of the light chain is the amino acid sequence set forth in SEQ ID NO: 13; or
(2) a first polypeptide chain comprising, from N-terminus to C-terminus, laIL-2, 1G4S, and an Fc in sequence, wherein laIL-2 is linked to the N-terminus of the Fc via 1G4S, and amino acid sequences of laIL-2 and the Fc are amino acid sequences set forth in SEQ ID NOs: 5 and 9, respectively; a second polypeptide chain comprising a heavy chain Fd fragment of the anti-PD-1 antibody and an Fc, wherein amino acid sequences of the heavy chain Fd fragment and the Fc are amino acid sequences set forth in SEQ ID NOs: 14 and 10, respectively; a third polypeptide chain comprising one light chain of the anti-PD-1 antibody, wherein an amino acid sequence of the light chain is the amino acid sequence set forth in SEQ ID NO: 13.

In some embodiments, the immunoconjugate comprises:
(1) a first polypeptide chain comprising the amino acid sequence set forth in SEQ ID NO: 17; a second polypeptide chain comprising the amino acid sequence set forth in SEQ ID NO: 18; a third polypeptide chain comprising the amino acid sequence set forth in SEQ ID NO: 13;
(2) a first polypeptide chain comprising the amino acid sequence set forth in SEQ ID NO: 19; a second polypeptide chain comprising the amino acid sequence set forth in SEQ ID NO: 20; two third polypeptide chains each comprising the amino acid sequence set forth in SEQ ID NO: 13;
(3) a first polypeptide chain comprising the amino acid sequence set forth in SEQ ID NO: 21; a second polypeptide chain comprising the amino acid sequence set forth in SEQ ID NO: 22; two third polypeptide chains each comprising the amino acid sequence set forth in SEQ ID NO: 13; or
(4) a first polypeptide chain comprising the amino acid sequence set forth in SEQ ID NO: 23; a second polypeptide chain comprising the amino acid sequence set forth in SEQ ID NO: 24; a third polypeptide chain comprising the amino acid sequence set forth in SEQ ID NO: 13.

In some preferred embodiments, the immunoconjugate comprises:
(1) a first polypeptide chain comprising the amino acid sequence set forth in SEQ ID NO: 21; a second polypeptide chain comprising the amino acid sequence set forth in SEQ ID NO: 22; two third polypeptide chains each comprising the amino acid sequence set forth in SEQ ID NO: 13; or
(2) a first polypeptide chain comprising the amino acid sequence set forth in SEQ ID NO: 25; a second polypeptide chain comprising the amino acid sequence set forth in SEQ ID NO: 26; a third polypeptide chain comprising the amino acid sequence set forth in SEQ ID NO: 13.

In a second aspect, the present disclosure also provides a nucleic acid encoding the aforementioned immunoconjugate.

In a third aspect, the present disclosure also provides a vector comprising the aforementioned nucleic acid.

In a fourth aspect, the present disclosure also provides a cell comprising the aforementioned nucleic acid or the aforementioned vector.

In a fifth aspect, the present disclosure also provides a pharmaceutical composition comprising the aforementioned immunoconjugate, the aforementioned nucleic acid, the aforementioned vector, and/or the aforementioned cell.

In some preferred embodiments, the pharmaceutical composition further comprises an additional therapeutic agent comprising a small-molecule drug, a nucleic acid drug, an antibody or an antibody fragment, and/or a polypeptide for use in treating cancer.

In some preferred embodiments, the pharmaceutical composition further comprises an excipient.

In a sixth aspect, the present disclosure provides use of the aforementioned immunoconjugate, the aforementioned nucleic acid, the aforementioned vector, the aforementioned cell, or the aforementioned pharmaceutical composition for preparing a medicament for preventing and/or treating cancer.

In a seventh aspect, the present disclosure provides a method for preventing and/or treating cancer in a subject in need thereof, comprising administering to the subject a therapeutically effective amount of the aforementioned immunoconjugate, the aforementioned nucleic acid, the aforementioned vector, the aforementioned cell, or the aforementioned pharmaceutical composition.

In some preferred embodiments, the cancer is a solid tumor, a hematologic tumor, or an anti-PD-1/PD-L1 antibody treatment-resistant cancer; the solid tumor is non-small cell lung cancer, small cell lung cancer, esophageal cancer, esophageal adenocarcinoma, gastric cancer, gastroesophageal junction cancer, colorectal cancer, microsatellite instability-high or mismatch repair-deficient colorectal cancer, microsatellite instability-high or mismatch repair-deficient cancer, melanoma, malignant pleural mesothelioma, cervical cancer, biliary tract cancer, cholangiocarcinoma, endometrial cancer, cancer with a high tumor mutational burden, cutaneous squamous cell carcinoma, hepatocellular carcinoma, pancreatic ductal adenocarcinoma, renal cell carcinoma, urothelial carcinoma, prostate cancer, ovarian cancer, breast cancer (triple-negative breast cancer), head and neck squamous cell carcinoma, nasopharyngeal carcinoma, glioblastoma, anaplastic thyroid carcinoma, Merkel cell carcinoma, or soft tissue sarcoma (synovial sarcoma); the hematologic tumor is Hodgkin lymphoma, classical Hodgkin lymphoma, lymphoma (diffuse large B-cell lymphoma, follicular lymphoma, or mantle cell lymphoma), primary mediastinal large B-cell lymphoma, leukemia (acute myeloid leukemia, chronic lymphocytic leukemia, or T-cell large granular lymphocytic leukemia), multiple myeloma, or myelofibrosis; the anti-PD-1/PD-L1 antibody treatment-resistant cancer is non-small cell lung cancer, microsatellite-stable colorectal cancer, hepatocellular carcinoma (β-catenin-activated type), pancreatic cancer with low PD-L1 expression, endometrial cancer, PTEN-deficient endometrial cancer, melanoma, melanoma progressing after previous anti-PD-1 treatment, a tumor rich in Treg or MDSC infiltration, or a tumor with activated WNT/β-catenin pathway.

In order to make the objectives, technical solutions, and advantages of the present disclosure clearer, the present disclosure will be further described in detail below with reference to the examples. Experimental procedures without specified conditions in the examples are conducted according to conventional conditions or conditions recommended by the manufacturer. Reagents or instruments without specified manufacturers are conventional products that are commercially available. Numerous specific details are set forth in the following specific examples in order to better explain the present disclosure. The specific examples described herein are merely illustrative of the present disclosure and are not intended to constitute any limitation on the present disclosure. In addition, descriptions of well-known structures and techniques are omitted in the following description to avoid unnecessarily obscuring the concepts of the present disclosure.

### EXAMPLE

### Example 1: Preparation of Immunoconjugates

### 1-1. Design of Immunoconjugates

The immunoconjugate was obtained by structural recombination from an antibody specifically binding to programmed death receptor 1 (an anti-PD-1 antibody) or an antigen-binding fragment thereof and low affinity IL-2 (laIL-2), and comprises the following three polypeptide chains:
1) a first polypeptide chain comprising or not comprising a heavy chain Fd fragment of the anti-PD-1 antibody, one single chain of an IgG1 Fc, a linker, and laIL-2;
2) a second polypeptide chain comprising a heavy chain Fd fragment of the anti-PD-1 antibody and one single chain of an IgG1 Fc;
3) a third polypeptide chain comprising a light chain of the anti-PD-1 antibody;
wherein the Fcs of the first polypeptide chain and the second polypeptide chain achieve heterodimerization through a Knob-into-Hole structure; one polypeptide chain of the Fc comprises T366W and S354C mutations (Fc knob chain), the other polypeptide chain comprises T366S, L368A, Y407V, and Y349C mutations (Fc hole chain), and the Fcs of the two polypeptide chains further comprise L234A and L235A mutations (LALA mutation) or L234A, L235A, and P329G mutations (LALAPG mutation).

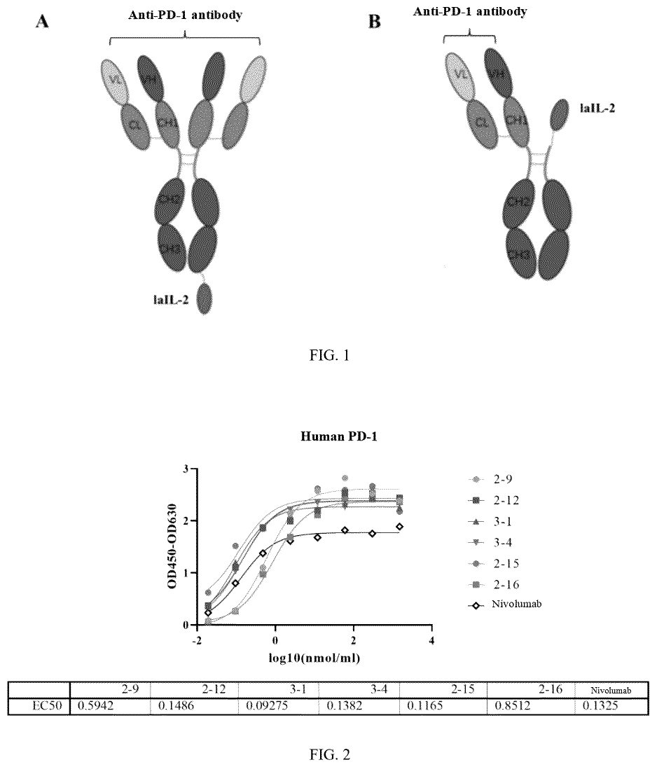

As shown in FIG. 1 and Table 1, a total of four immunoconjugates were designed, and the structures are as follows:

1) 2-9:
a first polypeptide chain (Knob chain) not comprising a heavy chain Fd fragment, but comprising, from N-terminus to C-terminus, laIL-2 with T3A, R38L, F42A, and C125S mutations, linker 1G4S, and an Fc knob chain with an LALA mutation in sequence, wherein laIL-2 is linked to the N-terminus of the Fc knob chain via 1G4S;
a second polypeptide chain (Hole chain) comprising an anti-PD-1 antibody heavy chain Fd fragment and an Fc hole chain with an LALA mutation;
a third polypeptide chain being an anti-PD-1 antibody light chain.
   2) 2-12:
a first polypeptide chain (Knob chain) comprising, from N-terminus to C-terminus, an anti-PD-1 antibody heavy chain Fd fragment, an Fc knob chain with an LALAPG mutation, 1G4S, and laIL-2 with T3A, R38L, F42A, and C125A mutations in sequence, wherein laIL-2 is linked to the C-terminus of the Fc knob chain via 1G4S;
a second polypeptide chain (Hole chain) comprising an anti-PD-1 antibody heavy chain Fd fragment and an Fc hole chain with an LALAPG mutation;
two third polypeptide chains, each third polypeptide chain being an anti-PD-1 antibody light chain.
   3) 2-15:
a first polypeptide chain (Knob chain) comprising, from N-terminus to C-terminus, an anti-PD-1 antibody heavy chain Fd fragment, an Fc knob chain with an LALAPG mutation, 1G4S, and laIL-2 with T3A, R38L, F42A, N88D, and C125A mutations in sequence, wherein laIL-2 is linked to the C-terminus of the Fc knob chain via 1G4S;
a second polypeptide chain (Hole chain) comprising an anti-PD-1 antibody heavy chain Fd fragment and an Fc hole chain with an LALAPG mutation;
two third polypeptide chains, each third polypeptide chain being an anti-PD-1 antibody light chain.
   4) 2-16:
a first polypeptide chain (Knob chain) not comprising a heavy chain Fd fragment, but comprising, from N-terminus to C-terminus, laIL-2 with T3A, R38L, F42A, N88D, and C125S mutations, 1G4S, and an Fc knob chain with an LALA mutation in sequence, wherein laIL-2 is linked to the N-terminus of the Fc knob chain via 1G4S;
a second polypeptide chain (Hole chain) comprising an anti-PD-1 antibody heavy chain Fd fragment and an Fc hole chain with an LALA mutation;
a third polypeptide chain being an anti-PD-1 antibody light chain.

The anti-PD-1 antibody fragments used in immunoconjugates 2-9, 2-12, 2-15, and 2-16 were derived from nivolumab.

TABLE 1
Sequences of immunoconjugates

| | | Light chain/heavy | | | |
|---|---|---|---|---|---|
| Molecule | | chain Fd (VH- | | | |
| No. | Polypeptide chain | CH1) | Fc(CH2-CH3) | Linker | laIL-2 |
| 2-9 | First polypeptide | / | SEQ ID NO: 9 | SEQ ID NO: 6 | SEQ ID NO: 2 |
| | chain | | | | |
| | Second | SEQ ID NO: 14 | SEQ ID NO: 10 | / | / |
| | polypeptide chain | | | | |
| | Third polypeptide | SEQ ID NO: 13 | / | / | / |
| | chain | | | | |
| 2-12 | First polypeptide | SEQ ID NO: 14 | SEQ ID NO: 11 | SEQ ID NO: 6 | SEQ ID NO: 4 |
| | chain | | | | |
| | Second | SEQ ID NO: 14 | SEQ ID NO: 12 | / | / |
| | polypeptide chain | | | | |
| | Third polypeptide | SEQ ID NO: 13 | / | / | / |
| | chain | | | | |
| 2-15 | First polypeptide | SEQ ID NO: 14 | SEQ ID NO: 11 | SEQ ID NO: 6 | SEQ ID NO: 3 |
| | chain | | | | |
| | Second | SEQ ID NO: 14 | SEQ ID NO: 12 | / | / |
| | polypeptide chain | | | | |
| | Third polypeptide | SEQ ID NO: 13 | / | / | / |
| | chain | | | | |
| 2-16 | First polypeptide | / | SEQ ID NO: 9 | SEQ ID NO: 6 | SEQ ID NO: 5 |
| | chain | | | | |
| | Second | SEQ ID NO: 14 | SEQ ID NO: 10 | / | / |
| | polypeptide chain | | | | |
| | Third polypeptide | SEQ ID NO: 13 | / | / | / |
| | chain | | | | |

In addition, immunoconjugates 3-1 and 3-4 were also prepared. Immunoconjugate 3-1 is an antibody fusion protein targeting the IL-2 receptor and PD-1, and its amino acid sequences are referenced in U.S. Patent No. US2018/0326010A1 and set forth in SEQ ID NO: 25, SEQ ID NO: 26, and SEQ ID NO: 15 in the present disclosure. Immunoconjugate 3-4 is a PD-1/IL-2a bispecific antibody fusion protein, and its amino acid sequences are referenced in International Patent No. WO2023/045977A1 and set forth in SEQ ID NO: 27, SEQ ID NO: 28, and SEQ ID NO: 16 in the present disclosure.

The specific sequence information of the above immunoconjugates is shown in Table 4.

### 1-2. Preparation

### 1. Molecular Sequence Synthesis

The amino acid sequence of the molecule was subjected to codon optimization for the Cricetulus griseus (CHO) expression system using the online design software on the GenScript website. The gene was then synthesized into the pEE6.4 vector by GenScript Biotech Corporation. The synthesized gene was subjected to plasmid extraction using NucleoBond Xtra Midi EF, Midi kit for endotoxin-free plasmid DNA (purchased from MACHEREY-NAGEL, Cat. No. 740420.50), and the plasmid was sent to Tsingke Biotech Co., Ltd. for sequencing and identification.

### 2. Protein Expression

1) HEK 293 cells were thawed in a water bath at 37 °C for 2 min and then transferred into 5 mL of Wayne293 LeTrans Medium (purchased from Zhongshan QuaCell, Cat. No. A21503). The mixture was centrifuged at 1000 rpm for 5 min, and the supernatant was discarded. The cells were resuspended in a certain amount of medium and counted using a counter. An appropriate amount of medium was added to adjust the cell density to (0.3-0.5)×10⁶ cells/mL. The cells were cultured with shaking in a shaker, with the shaker set at the following parameters: a temperature of 37 °C, 5% CO₂, and a rotation speed of 120 rpm. The cells can be passaged once 2-3 days after expansion, and the thawed cells should be passaged at least three times before other experiments.
2) Cell preparation before transfection: The cell density and viability were measured on the day of transfection. The cell density was adjusted to 3×10⁶ cells/mL with Wayne293® LeTrans Medium, and the viability was ≥95%. The cells were gently shaken uniformly and aliquoted into shake flasks. The flasks were placed in a shaker for later use.
3) Transient transfection: The amount of transfection DNA was 1 μg/mL, and the ratio of DNA to polyethylenimine (Polyethylenimine L, PEI; purchased from Polyscience, Cat. No. 24765-1) was 1:4. Two 50 mL centrifuge tubes were taken. A volume of Opti-MEM reduced serum medium (purchased from Gibco, Cat. No. 31985070) equivalent to 3% of the transfection volume was added to each tube. DNA to be transfected and a PEI transfection reagent were added separately to the two tubes of Opti-MEM reduced serum medium. The mixtures were thoroughly mixed uniformly and left to stand for 5 min, designated as component A and component B, respectively. B was added dropwise to A, and the mixture was thoroughly mixed uniformly and left to stand at room temperature for 15 min. Finally, the DNA-PEI mixture was added dropwise to the aliquoted cells. A feed supplement was performed 22-24 h after transfection by adding Wayne293 LeTrans Feed Medium (purchased from Zhongshan QuaCell, Cat. No. A21901) at a volume equivalent to 9% of the transfection system volume. The culture was terminated when the cell viability was less than 60% or the expression amount no longer showed a significant increase.
4) Harvest of transfection supernatant: The cell supernatant after transient transfection was centrifuged at 5000 rpm and filtered through a 0.22 μm filter membrane, and then the purification process was performed.

### 3. Protein Purification

Except that the affinity chromatography step of molecule 2-15 was performed using isocratic elution, other molecules were prepared using linear elution. After elution and collection were completed, the SEC purity of each component was detected, and the components with higher purity were pooled as the collected affinity-eluted sample. After affinity chromatography, the protein was subjected to anion exchange chromatography to remove impurities such as cell host proteins.

### Example 2: Affinity Assay

The binding abilities of immunoconjugates to PD-1, IL-2Rα, and IL-2Rβγ were detected by enzyme-linked immunosorbent assay (Elisa), and the difference in affinity of the protein molecules for PD-1, IL-2Rα, and the IL-2Rβγ heterodimer was evaluated.

### 2-1. Binding Abilities of Immunoconjugates to PD-1

### 1. Antigen Coating

A recombinant human PD-1 protein (purchased from Sino Biological, Cat. No. 10377-H08H) was diluted to a concentration of 5 μg/mL and added to a 96-well microplate at 100 μL/well. The plate was coated at 4 °C overnight.

### 2. Blocking Treatment

The liquid in the wells was discarded, and the plate was washed 4 times with phosphate buffered saline (PBS, pH 7.4) at 250 μL/well. A blocking buffer containing 2% bovine serum albumin (BSA) was added at 200 μL/well, and the plate was blocked in an incubator at 37 °C for 2 h.

### 3. Sample Incubation

The test immunoconjugates and the positive control antibody nivolumab (purchased from Sino Biological, Cat. No. 68050-H001) were serially diluted in a 1:5 ratio from an initial concentration of 1500 nmol/mL to prepare 8 concentration gradients. Samples of each concentration were added to the corresponding wells at 100 μL/well and incubated at a constant temperature of 37 °C for 1 h.

### 4. Secondary Antibody Reaction

The liquid in the wells was discarded, and the plate was washed 4 times with a Tris-HCl buffer (TBST) containing 0.05% Tween-20 at 250 μL/well.

A horseradish peroxidase (HRP)-labeled anti-human IgG (Fc fragment) secondary antibody (purchased from Sigma-Aldrich, Cat. No. A0170) diluted in a 1:10000 ratio was added at 100 μL/well, and the mixture was incubated at 37 °C for 1 h in the dark.

### 5. Color Development Detection

The liquid in the wells was discarded, and the plate was washed 4 times with TBST at 250 μL/well.

A TMB chromogenic substrate was added at 100 μL/well, and the mixture was reacted at room temperature for 20-40 min in the dark.

100 μL of stop solution was added to stop the reaction, and the absorbance values at a wavelength of OD 450 nm-OD 630 nm were immediately measured using a microplate reader.

The results show that the binding curves of the target immunoconjugates to PD-1 showed a binding trend similar to that of the control antibody nivolumab. Under the equimolar concentration conditions, the binding activities of 2-9 and 2-16 showed a relatively decreasing trend as compared to the control antibody. The difference is related to the number of Fab domains in the molecular structure (FIG. 2).

### 2-2. Binding Abilities of Immunoconjugates to IL-2Rα and IL-2Rβγ

The experimental method was the same as that described in Example 2-1, except that the coating antibody recombinant human PD-1 protein was replaced with a recombinant human IL-2Rα protein (Acro Biosystems, Cat. No. ILA-H52H9) and an IL-2Rβγ heterodimer protein (Acro Biosystems, Cat. No. ILG-H5283), and the positive control antibody nivolumab was replaced with wild-type IL-2.

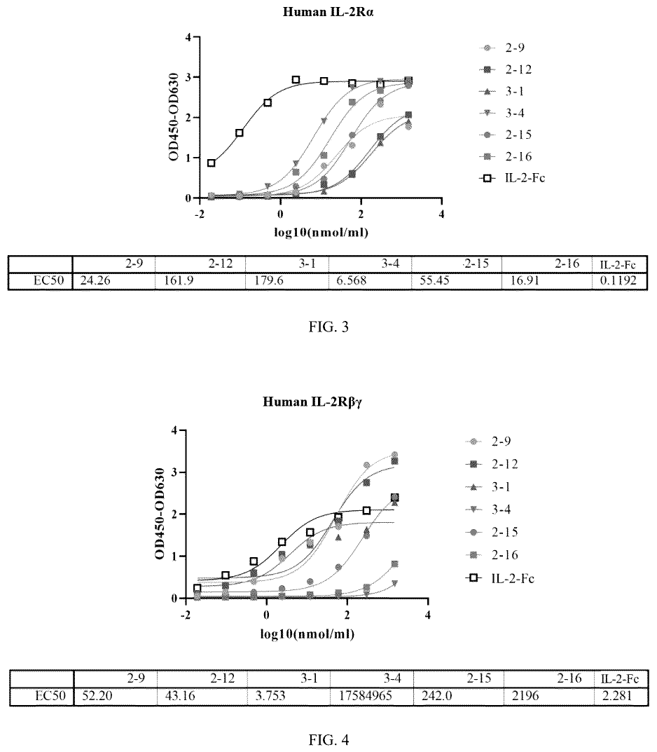

The results show that the binding activities of 3-1, 2-15, and 2-16 to IL-2Rα were significantly reduced as compared to that of the control antibody, while 3-4 maintained the binding activity to IL-2Rα; meanwhile, the binding activities of 3-4, 2-15, and 2-16 to IL-2Rβγ also showed a decreasing trend (FIGS. 3 and 4).

### Example 3: Cell Binding Activity

In this example, fluorescence activated cell sorting (FACS) was used. HEK-blue-IL-2 cells (purchased from InvivoGen) were used to detect the binding of IL-2 in the immunoconjugates to human IL-2R, and HEK-blue-IL-2/hPD-1 cells (purchased from InvivoGen) were used to detect the affinity of the immunoconjugates for PD-1. The HEK-blue-IL-2/hPD-1 cells were obtained by transfecting HEK-blue-IL-2 cells with a human PD-1 expression plasmid packaged by a lentivirus to achieve overexpression of hPD-1, and clones with stable expression were selected for detecting the binding activity of each immunoconjugate to human PD-1 on the surface of HEK-Blue™-IL-2/hPD-1 cells. Specifically, the following steps are included:

### 1. Preparation of Cell Samples

HEK-blue-IL-2 cells (solely expressing the IL-2 receptor) and HEK-blue-IL-2/hPD-1 cells (co-expressing the IL-2 receptor and human PD-1) were separately seeded in a 96-well plate at a density of 3× 10⁵ cells/well. 100 μL of cell suspension was added to each well. The mixture was centrifuged at 2000 rpm for 5 min, and the supernatant was discarded.

### 2. Sample Incubation and Serial Dilution

The test immunoconjugates and the control antibody IL-2-Fc/nivolumab were serially diluted in a 1:5 ratio from an initial concentration of 300 nmol/mL to prepare 8 concentration gradients. 100 μL of each concentration gradient sample was taken, added to the corresponding wells, and incubated with the cells at 4 °C for 30 min. After the binding reaction was completed, the cells were washed once with 200 μL of FACS buffer.

### 3. Secondary Antibody Labeling and Detection

0.3 μL/well of PE-labeled anti-human IgG Fc fragment secondary antibody (purchased from BioLegend) was taken, and after dilution, the cell suspension was added at 100 μL/well. The mixture was incubated at 4 °C for 30 min in the dark. The cells were washed twice with 200 μL of FACS buffer and finally resuspended in 200 μL of FACS buffer. The PE fluorescence signal intensity was detected using a flow cytometer, and the binding activity of the target protein to the cell surface receptor was analyzed.

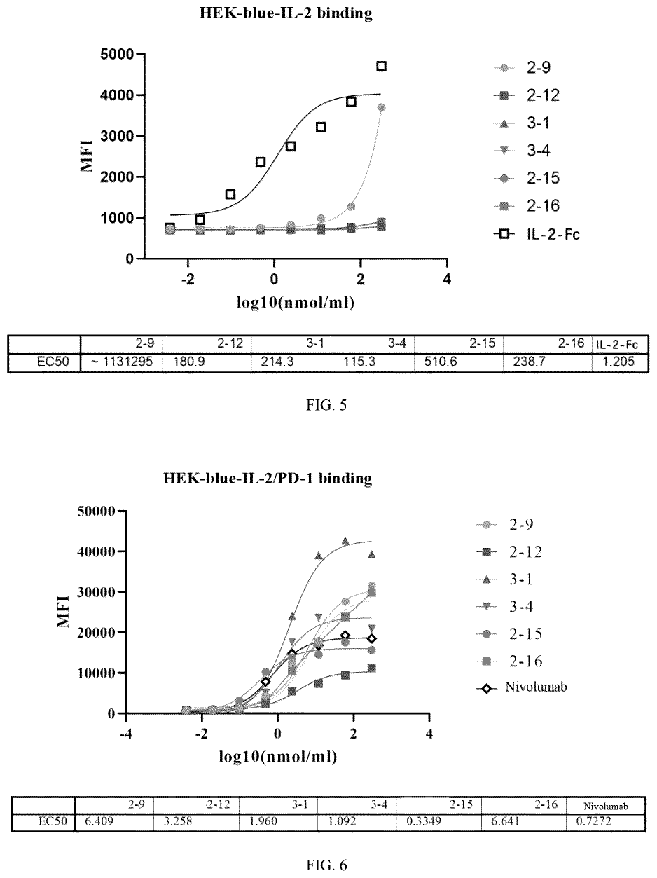

The results show that in HEK-blue-IL-2 cells, no significant binding activity of the immunoconjugates to the IL-2 receptor was detected; however, in HEK-blue-IL-2/PD-1 cells, the binding activities of the immunoconjugates to the cells were restored, and the binding characteristics of the immunoconjugates were associated with the PD-1-targeting function (FIGS. 5 and 6).

### Example 4: IL-2 Activity in Immunoconjugates

In this example, the activity of IL-2 in the immunoconjugates was detected using HEK-blue-IL-2/HEK-blue-IL-2/hPD-1 cells. The specific steps were as follows:

### 1. Cell Seeding and Treatment

HEK-blue-IL-2 cells and HEK-blue-IL-2/PD-1 cells were separately seeded in a 96-well plate at a density of 1.5× 10⁵ cells/well, and 100 μL of cell suspension was added to each well.

### 2. Sample Serial Dilution and Co-Culture

The test immunoconjugates and the control protein were diluted to an initial concentration of 300 nmol/mL with a DMEM high glucose medium (M&C Gene Technology Ltd., Cat. No. CM10013), and concentration gradients were successively prepared by a five-fold serial dilution method. The samples with a concentration of 60 nmol/mL were selected for the assay, added to the corresponding cell wells at 100 μL/well, and co-cultured with the cells in an incubator at 37 °C with 5% CO₂ for 24 h.

### 3. Reporter Gene Activity Detection

After the co-culture was completed, 20 μL of the supernatant from each well was taken and transferred into a new 96-well plate, and 180 μL of QUANTI-Blue™ detection solution (purchased from Invivogen) was added. The mixture was mixed uniformly and incubated at 37 °C for 1 h in the dark. The absorbance values at a wavelength of 630 nm were measured using a microplate reader.

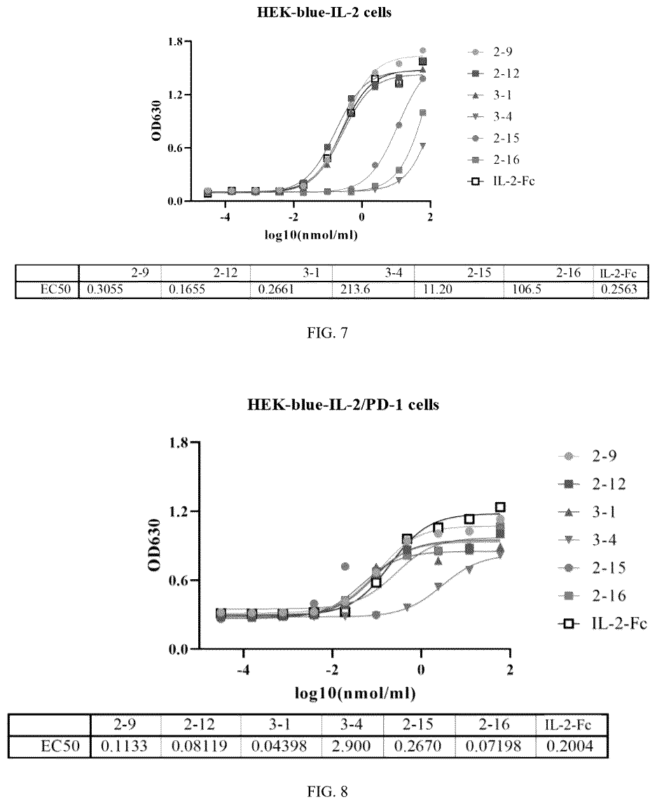

The results show that in HEK-blue-IL-2 cells, the IL-2 signal activation levels of immunoconjugates 2-15 and 2-16 were significantly reduced, demonstrating a significant difference as compared to those of 2-9 and 2-12; in HEK-blue-IL-2/PD-1 cells, the IL-2 signal activation levels of 2-15 and 2-16 were restored, and the enhanced activities of the immunoconjugates were associated with the expression of PD-1 (FIGS. 7 and 8).

### Example 5: Activation of Downstream Signals by Immunoconjugates Comprising IL-2

In this example, the activation of downstream signals by immunoconjugates comprising IL-2 was detected using unactivated PBMC (Naïve PBMC) cells. The specific procedures were as follows:

### 1. Cell Pretreatment and Surface Labeling

Naïve PBMC cells (purchased from MileCell Biotechnology, Cat. No. PB100C) were taken and seeded in a 96-well plate at a density of 1×10⁶ cells/well. The test immunoconjugates (2-9, 2-12, 3-1, 3-4, and 2-15/16) and the control protein IL-2-Fc (IL-2 was wild-type) were serially diluted in a 1:5 ratio to prepare 8 concentration gradients.

The cells were firstly stained with a live/dead dye for 30 min and then washed once with 200 μL of pre-heated RPMI-1640 (purchased from MACGENE, Cat. No. CM10040) complete medium; the protein diluted with the pre-heated medium was added in a corresponding gradient, and the mixture was incubated in a water bath at 37 °C for 20 min.

### 2. Cell Fixation and Intracellular Staining

After the reaction was terminated, 200 μL of pre-heated fixation buffer (purchased from BD Biosciences, Cat. No. 554655) was added to each well, and the mixture was mixed uniformly and left to stand at 37 °C for 10 min.

### 3. FACS Assay

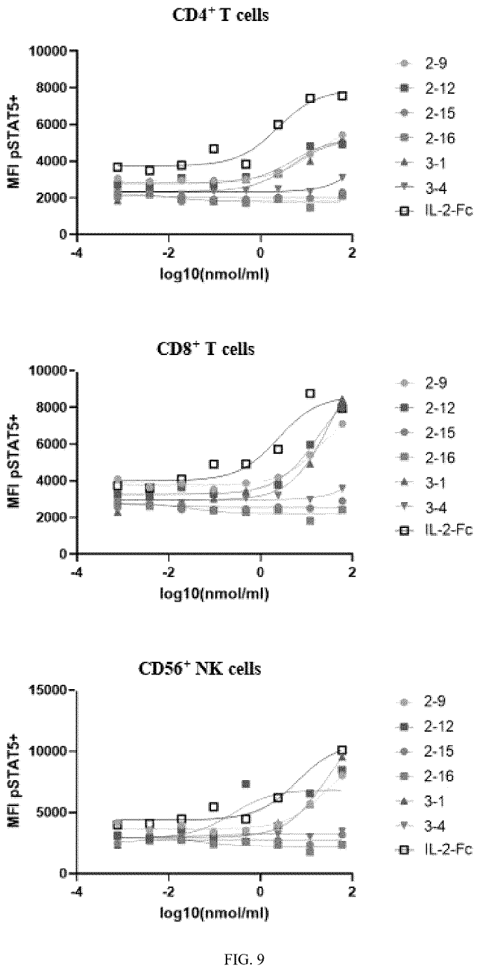

The cells were washed once with 200 μL of FACS buffer, resuspended in 200 μL of FACS buffer, and transferred into a flow cytometry tube. The mixture was centrifuged, and the supernatant was discarded. 1 mL of pre-cooled permeabilization buffer (Perm buffer III, purchased from BD Biosciences, Cat. No. 558050) was added while shaking, and the mixture was incubated on ice for 30 min. After the permeabilization was completed, the cells were washed twice with an FACS buffer, and a mixture of antibodies (including a BV711-labeled anti-human CD56 antibody (purchased from BioLegend, Cat. No. 318336), an FITC-labeled anti-human CD8 antibody (purchased from BioLegend, Cat. No. 300906), a PE-labeled anti-human CD25 antibody (purchased from BioLegend, Cat. No. 356104), an APC-labeled anti-human pSTAT5 antibody (purchased from BioLegend, Cat. No. 936906), and an AF700-labeled anti-human CD4 antibody (purchased from BioLegend, Cat. No. 300526)) was added. The resulting mixture was incubated for 1 h. After the incubation was completed, the cells were washed one more time with an FACS buffer, and an FACS buffer was added to resuspend the cells for the assay. The results show that compared to IL-2-Fc, immunoconjugates 2-9, 2-12, and 3-1 were still able to activate Naïve CD4+ T cells, CD8+ T cells, and NK cells in human peripheral blood with a relatively low binding activity, which indicates that immunoconjugates 2-9, 2-12, and 3-1 have potential in vivo toxicity. In contrast, no significant activation of naive T cells and NK cells in human peripheral blood was detected for immunoconjugates 2-15, 2-16, and 3-4, which suggests that these immunoconjugates have relatively low in vivo toxicity (FIG. 9).

### Example 6: Cell Death and Proliferation within 1-7 Days of Co-Incubation of Immunoconjugates Comprising IL-2 and Receptor

In this example, the cell death and cell proliferation within 1-7 days of the co-incubation of immunoconjugates comprising IL-2 and a receptor were detected using Naïve PBMC cells. The specific procedures were as follows:

### 1. Cell Culture and Treatment

Naïve PBMC cells were taken and seeded in a 96-well plate at a density of 1×10⁶ cells/well. The initial concentration of the immunoconjugate was 60 μmol/L, and meanwhile, a CD3/CD28 antibody stimulation group was set as a positive control. A mixed solution of 250 μL of cell suspension and 250 μL of medium containing the immunoconjugate was added to each well. The cells were continuously cultured in an incubator at 37 °C with 5% CO₂ for 1-7 days, and samples were collected on days 1, 3, 5, and 7 of the co-culture for analysis.

### 2. Cell Death Detection

Cell suspensions at each time point were collected and centrifuged. The cells were taken, washed once with 200 μL of FACS buffer, and resuspended. 2 μL of 7-AAD dye was added to observe cell death.

### 3. FACS Assay

Cells at each time point were collected, and an AF700-labeled anti-human CD4 antibody (purchased from BioLegend, Cat. No. 300526) and an FITC-labeled anti-human CD8 antibody (purchased from BioLegend, Cat. No. 300906) were separately added to the cells. After 30 min of staining, the cells were washed once with 200 μL of FACS buffer. The cells were fixed and permeabilized with pre-cooled 70% ethanol for 1 h. After the supernatant was discarded, an APC-labeled anti-human Ki67 antibody (purchased from BioLegend, Cat. No. 350514) was added at 2 μL/well, and the mixture was incubated on ice for 30 min. The cells were washed once with 200 μL of FACS buffer, and an FACS buffer was added to resuspend the cells for the assay.

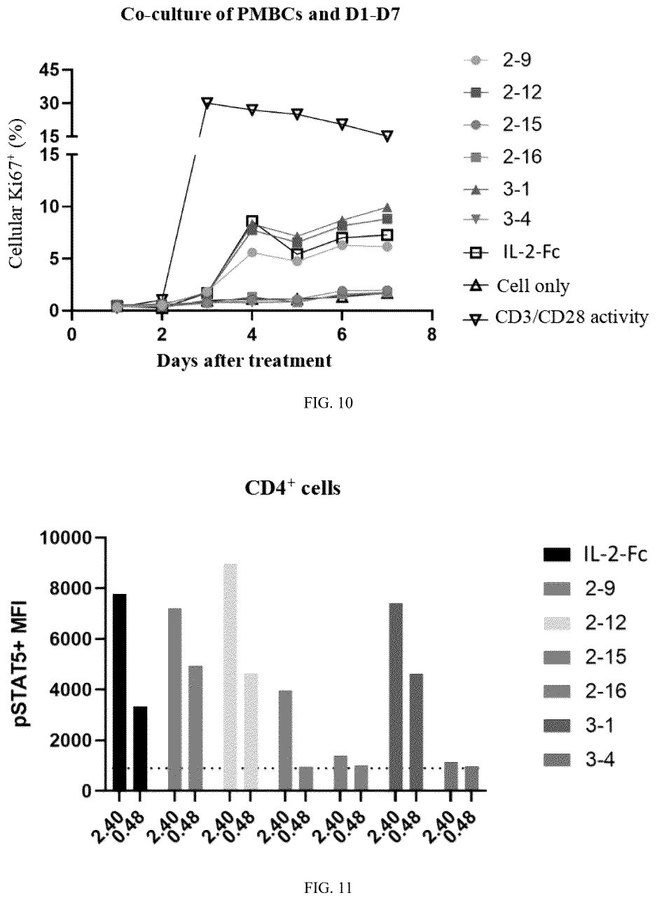

The results show that after immunoconjugates 2-15, 2-16, and 3-4 were co-incubated with human PBMCs in vitro for a long time, no activation of PBMCs by the immunoconjugates was observed, which suggests that these immunoconjugates have good safety (FIG. 10).

### Example 7: Activation of Downstream Signals by Immunoconjugates Comprising IL-2

In this example, the activation of downstream signals by immunoconjugates comprising IL-2 was detected using CD3/CD28-activated PBMC cells. The specific steps were as follows:

### 1. Cell Treatment and Labeling

PBMC cells activated with CD3/CD28 were seeded in a 96-well plate at a density of 1× 10⁶ cells/well. The test immunoconjugates and the control protein were serially diluted in a 1:5 ratio from an initial concentration of 2.4 nmol/mL to prepare 2 concentration gradients. A live/dead dye (yellow fluorescent active dye, purchased from Invitrogen, Cat. No. 2622316) was added for staining for 30 min, and the plate was washed once with a pre-heated RPMI-1640 complete medium. The test immunoconjugate diluted with the pre-heated RPMI-1640 complete medium was added and incubated in a water bath at 37 °C for 25 min.

### 2. Cell Fixation and Intracellular Staining

200 μL of pre-heated fixation buffer (purchased from BD Biosciences, Cat. No. 554655) was added, and the mixture was mixed uniformly and left to stand at 37 °C for 10 min. The cells were washed once with 200 μL of FACS buffer, resuspended in 200 μL of FACS buffer, and then transferred into a flow cytometry tube. The mixture was centrifuged, and the supernatant was discarded. 1 mL of pre-cooled permeabilization buffer (BD Biosciences, Cat. No. 558050) was added while shaking, and the mixture was incubated on ice for 30 min. After centrifugation, the supernatant was discarded. The cells were washed twice with 200 μL of FACS buffer, and a mixture of antibodies (a BV711-labeled anti-human CD56 antibody, an FITC-labeled anti-human CD8 antibody, an APC-labeled anti-human pSTAT5 antibody, and an AF700-labeled anti-human CD4 antibody) was added, followed by 1 h of staining.

### 3. FACS Assay

The cells were washed once with an FACS buffer and resuspended in an FACS buffer for the assay.

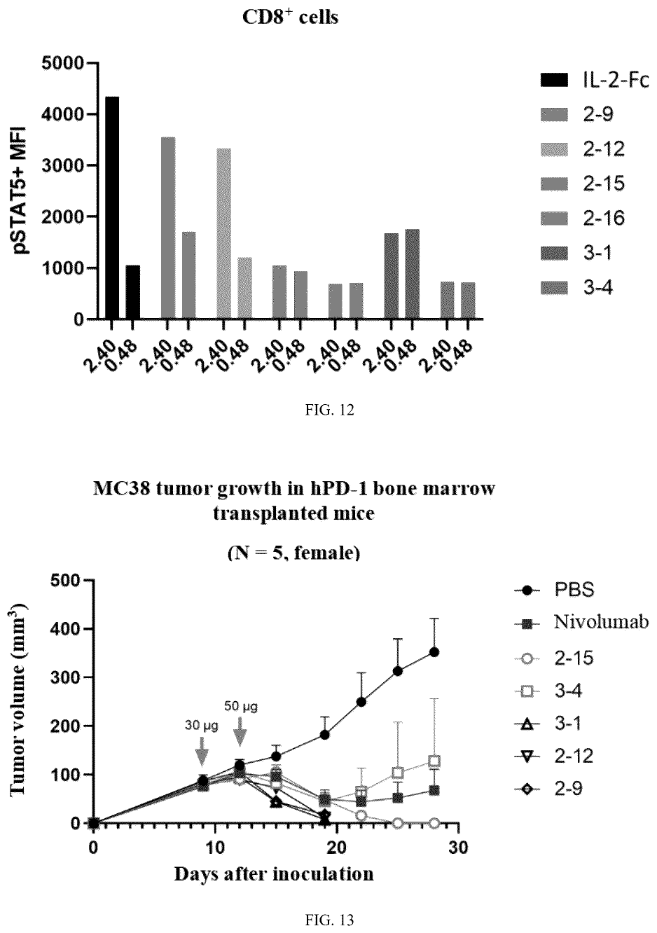

The experimental results show that immunoconjugate 2-15 has a relatively good activation effect on the activated T cells after binding (FIGS. 11 and 12).

### Example 8: Anti-Tumor Effects of Immunoconjugates Comprising IL-2

In this example, a subcutaneous tumor model was established using MC38 cells, and the anti-tumor effects of the candidate molecules were tested in vivo. The specific steps were as follows:

### 1. Establishment of Tumor Model

C57BL/6J-Pdcdlem1 (hPDCD1)/Smoc mice (purchased from Shanghai Model Organisms Center, Inc., Cat. No. NM-HU-00015) were used as donor mice for bone marrow transplantation. C57BL/6 mice (purchased from Vital River, Cat. No. 213) were inoculated with MC38 tumors to detect the in vivo tumor-inhibiting activity of the target proteins. MC38 colon cancer cells in the logarithmic growth phase (viability>90%) were taken and subcutaneously injected into the mice at an inoculation amount of 1×10⁶ cells/mouse; the growth rate of the tumors was monitored, and the mice were randomly grouped when the tumors grew to 80-100 mm³.

### 2. Design of Administration Regimen

Tumor-bearing mice were divided into 7 groups (n=5/group), and the administration regimen for each group is shown in Table 2.

The treatment groups were given different fusion drugs (Table 2) by intraperitoneal injection twice a week at an interval of 72 h.

TABLE 2
Administration regimen

| | | | | Mode of | Frequency of |
|---|---|---|---|---|---|
| Group | Number | Treatment | Dose | injection | administration |
| 1 | 5 | PBS | Equivalent volume × 2 | Intraperitoneal | Twice a week |
| 2 | 5 | 2-9 | 30 μg + 50 μg | Intraperitoneal | Twice a week |
| 3 | 5 | 2-12 | 30 μg + 50 μg | Intraperitoneal | Twice a week |
| 4 | 5 | 2-15 | 30 μg + 50 μg | Intraperitoneal | Twice a week |
| 5 | 5 | 3-1 | 30 μg + 50 μg | Intraperitoneal | Twice a week |
| 6 | 5 | 3-4 | 30 μg + 50 μg | Intraperitoneal | Twice a week |
| 7 | 5 | Nivolumab | 30 μg + 50 μg | Intraperitoneal | Twice a week |

### 3. Drug Effect and Safety Monitoring

The tumor volume was measured every 3-4 days, and simultaneously, the mice were weighed.

### 4. Experimental Results

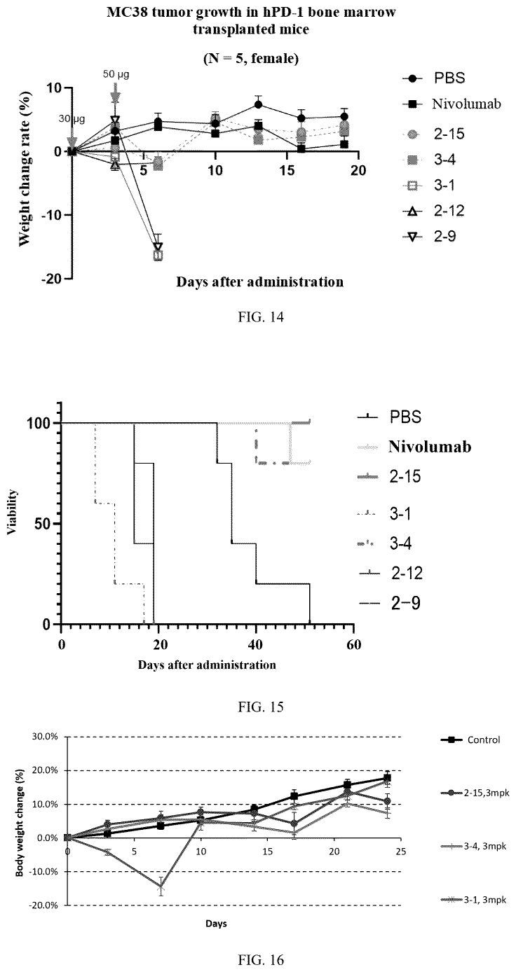

As can be seen from the experimental results, compared to the control treatment groups, the group treated with 2-15 achieved a 100% rate of complete tumor regression in the experimental animals, exhibiting a significant anti-tumor effect (FIG. 13). No significant weight loss was observed in the mice (FIG. 14). In contrast, groups treated with immunoconjugates 3-1, 2-9, and 2-12 showed significant weight loss after the second administration, which was accompanied by partial death of the mice (FIG. 15). All the in vivo experimental results show that immunoconjugate 2-15 had a significant drug effect and exhibited no significant toxic and side effects at this dose.

### Example 9: Efficacy of Immunoconjugates Comprising IL-2 in a Mouse Pancreatic Cancer Model

Pan02 pancreatic cancer cells in the logarithmic growth phase were subcutaneously injected into hPD-1 HuGEMM (C57BL/6) mice (Biocytogen) at an inoculation amount of 3× 10⁶ cells/mouse. Tumor growth was monitored, and the mice were randomly divided into 4 groups (6 mice/group) when the average tumor size reached 80-110 mm³. The grouping day was recorded as Day 0 (DO). Immunoconjugates 2-15, 3-1, or 3-4 was administered via intraperitoneal injection twice a week at 3 mpk, while the control group received an equal volume of 0.9% sodium chloride injection. Tumor volumes were measured to calculate the Tumor Growth Inhibition rate (TGI), and mouse body weights were weighed.

TGI %=(1−Ti/Ci)×100%, where Ti and Ci are the average tumor volumes of the treatment group and control group at a specific time point, respectively.

One mouse in the 3-1 treatment group died on D7. 2-15 exhibited good safety and superior anti-tumor efficacy to 3-1 and 3-4 in the mouse pancreatic cancer model as shown in Table 3 and FIG. 16.

TABLE 3
Tumor Growth Inhibition rate on D 24

| Group | TGI(%) |
|---|---|
| 2-15 | 90.6 |
| 3-1 | 78.5 |
| 3-4 | 80.0 |

The various technical features of the above examples may be combined arbitrarily. In order to make the description brief, not all possible combinations of the various technical features in the above examples are described. However, as long as there is no contradiction in the combinations of the technical features, it should be considered as being within the scope of the present specification.

The above examples only illustrate several embodiments of the present disclosure for the purpose of specific and detailed description, but should not be construed as limiting the scope of the present disclosure. It should be noted that various changes and modifications can be made by those of ordinary skills in the art without departing from the concept of the present disclosure, and these changes and modifications are all within the protection scope of the present disclosure. Therefore, the protection scope of the present patent disclosure shall be subject to the appended claims.

TABLE 4
Sequence information

| | | SEQ ID |
|---|---|---|
| Sequence name | Sequence | NO: |
| Wild-type IL-2 | APTSSSTKKTQLQLEHLLLDLQMILNGINNYKNPKLTRMLTFKFYMP | 1 |
| | KKATELKHLQCLEEELKPLEEVLNLAQSKNFHLRPRDLISNINVIVLE | |
| | LKGSETTFMCEYADETATIVEFLNRWITFCQSIISTLT | |
| Immunoconjugate | APASSSTKKTQLQLEHLLLDLQMILNGINNYKNPKLTLMLTAKFYMP | 2 |
| 2-9 1aIL-2 | KKATELKHLQCLEEELKPLEEVLNLAQSKNFHLRPRDLISNINVIVLE | |
| | LKGSETTFMCEYADETATIVEFLNRWITFSQSIISTLT | |
| Immunoconjugate | APASSSTKKTQLQLEHLLLDLQMILNGINNYKNPKLTLMLTAKFYMP | 3 |
| 2-15 1aIL-2 | KKATELKHLQCLEEELKPLEEVLNLAQSKNFHLRPRDLISDINVIVLE | |
| | LKGSETTFMCEYADETATIVEFLNRWITFAQSIISTLT | |
| Immunoconjugate | APASSSTKKTQLQLEHLLLDLQMILNGINNYKNPKLTLMLTAKFYMP | 4 |
| 2-12 1aIL-2 | KKATELKHLQCLEEELKPLEEVLNLAQSKNFHLRPRDLISNINVIVLE | |
| | LKGSETTFMCEYADETATIVEFLNRWITFAQSIISTLT | |
| Immunoconjugate | APASSSTKKTQLQLEHLLLDLQMILNGINNYKNPKLTLMLTAKFYMP | 5 |
| 2-16 1aIL-2 | KKATELKHLQCLEEELKPLEEVLNLAQSKNFHLRPRDLISDINVIVLE | |
| | LKGSETTFMCEYADETATIVEFLNRWITFSQSIISTLT | |
| Linker 1G4S | GGGGS | 6 |
| Linker 2G4S | GGGGSGGGGS | 7 |
| Linker 3G4S | GGGGSGGGGSGGGGS | 8 |
| Fc knob chain of 2- | APEAAGGPSVFLFPPKPKDTLMISRTPEVTCVVVDVSHEDPEVKFN | 9 |
| 9 and 2-16 (LALA | WYVDGVEVHNAKTKPREEQYNSTYRVVSVLTVLHQDWLNGKEYK | |
| mutation) | CKVSNKALPAPIEKTISKAKGQPREPQVYTLPPCRDELTKNQVSLWC | |
| | LVKGFYPSDIAVEWESNGQPENNYKTTPPVLDSDGSFFLYSKLTVDK | |
| | SRWQQGNVFSCSVMHEALHNHYTQKSLSLSPGK | |
| Fc hole chain of 2- | APEAAGGPSVFLFPPKPKDTLMISRTPEVTCVVVDVSHEDPEVKFN | 10 |
| 9 and 2-16 (LALA | WYVDGVEVHNAKTKPREEQYNSTYRVVSVLTVLHQDWLNGKEYK | |
| mutation) | CKVSNKALPAPIEKTISKAKGQPREPQVCTLPPSRDELTKNQVSLSCA | |
| | VKGFYPSDIAVEWESNGQPENNYKTTPPVLDSDGSFFLVSKLTVDKS | |
| | RWQQGNVFSCSVMHEALHNHYTQKSLSLSPGK | |
| Fc knob chain of 2- | APEAAGGPSVFLFPPKPKDTLMISRTPEVTCVVVDVSHEDPEVKFN | 11 |
| 12 and 2-15 | WYVDGVEVHNAKTKPREEQYNSTYRVVSVLTVLHQDWLNGKEYK | |
| (LALAPG | CKVSNKALGAPIEKTISKAKGQPREPQVYTLPPCRDELTKNQVSLWC | |
| mutation) | LVKGFYPSDIAVEWESNGQPENNYKTTPPVLDSDGSFFLYSKLTVDK | |
| | SRWQQGNVFSCSVMHEALHNHYTQKSLSLSPG | |
| Fc hole chain of Fc | APEAAGGPSVFLFPPKPKDTLMISRTPEVTCVVVDVSHEDPEVKFN | 12 |
| fusion proteins 2- | WYVDGVEVHNAKTKPREEQYNSTYRVVSVLTVLHQDWLNGKEYK | |
| 12 and 2-15 | CKVSNKALGAPIEKTISKAKGQPREPQVCTLPPSRDELTKNQVSLSC | |
| (LALAPG | AVKGFYPSDIAVEWESNGQPENNYKTTPPVLDSDGSFFLVSKLTVDK | |
| mutation) | SRWQQGNVFSCSVMHEALHNHYTQKSLSLSPGK | |
| Anti-PD-1 antibody | EIVLTQSPATLSLSPGERATLSCRASQSVSSYLAWYQQKPGQAPRLLI | 13 |
| light chain of | YDASNRATGIPARFSGSGSGTDFTLTISSLEPEDFAVYYCQQSSNWPR | |
| immunoconjugates | TFGQGTKVEIKRTVAAPSVFIFPPSDEQLKSGTASVVCLLNNFYPREA | |
| 2-9, 2-12, 2-15, and | KVQWKVDNALQSGNSQESVTEQDSKDSTYSLSSTLTLSKADYEKH | |
| 2-16 | KVYACEVTHQGLSSPVTKSFNRGEC | |
| Anti-PD-1 antibody | QVQLVESGGGVVQPGRSLRLDCKASGITFSNSGMHWVRQAPGKGL | 14 |
| heavy chain Fd | EWVAVIWYDGSKRYYADSVKGRFTISRDNSKNTLFLQMNSLRAEDT | |
| fragment of | AVYYCATNDDYWGQGTLVTVSSASTKGPSVFPLAPSSKSTSGGTAA | |
| immunoconjugates | LGCLVKDYFPEPVTVSWNSGALTSGVHTFPAVLQSSGLYSLSSVVTV | |
| 2-9, 2-12, 2-15, and | PSSSLGTQTYICNVNHKPSNTKVDKKV | |
| 2-16 | | |
| Anti-PD-1 antibody | DIVMTQSPDSLAVSLGERATINCKASESVDTSDNSFIHWYQQKPGQS | 15 |
| light chain of | PKLLIYRSSTLESGVPDRFSGSGSGTDFTLTISSLQAEDVAVYYCQQN | |
| immunoconjugate | YDVPWTFGQGTKVEIKRTVAAPSVFIFPPSDEQLKSGTASVVCLLNN | |
| 3-1 | FYPREAKVQWKVDNALQSGNSQESVTEQDSKDSTYSLSSTLTLSKA | |
| | DYEKHKVYACEVTHQGLSSPVTKSFNRGEC | |
| Anti-PD-1 antibody | DIQMTQSPSSVSASVGDRVTITCRASQGISSWLAWYQQKPGKAPKLL | 16 |
| light chain of | ISAASSLQSGVPSRFSGSGSGTDFTLTISSLQPEDFATYYCQQANHLPF | |
| immunoconjugate | TFGGGTKVEIKRTVAAPSVFIFPPSDEQLKSGTASVVCLLNNFYPREA | |
| 3-4 | KVQWKVDNALQSGNSQESVTEQDSKDSTYSLSSTLTLSKADYEKH | |
| | KVYACEVTHQGLSSPVTKSFNRGEC | |
| First polypeptide | APASSSTKKTQLQLEHLLLDLQMILNGINNYKNPKLTLMLTAKFYMP | 17 |
| chain of | KKATELKHLQCLEEELKPLEEVLNLAQSKNFHLRPRDLISNINVIVLE | |
| immunoconjugate | LKGSETTFMCEYADETATIVEFLNRWITFSQSIISTLTGGGGSDKTHTC | |
| 2-9 | PPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNGK | |
| | EYKCKVSNKALPAPIEKTISKAKGQPREPQVYTLPPCRDELTKNQVS | |
| | LWCLVKGFYPSDIAVEWESNGQPENNYKTTPPVLDSDGSFFLYSKLT | |
| | VDKSRWQQGNVFSCSVMHEALHNHYTQKSLSLSPGK | |
| Second polypeptide | QVQLVESGGGVVQPGRSLRLDCKASGITFSNSGMHWVRQAPGKGL | 18 |
| chain of | EWVAVIWYDGSKRYYADSVKGRFTISRDNSKNTLFLQMNSLRAEDT | |
| immunoconjugate | AVYYCATNDDYWGQGTLVTVSSASTKGPSVFPLAPSSKSTSGGTAA | |
| 2-9 | LGCLVKDYFPEPVTVSWNSGALTSGVHTFPAVLQSSGLYSLSSVVTV | |
| | PSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSN | |
| | KALPAPIEKTISKAKGQPREPQVCTLPPSRDELTKNQVSLSCAVKGFY | |
| | PSDIAVEWESNGQPENNYKTTPPVLDSDGSFFLVSKLTVDKSRWQQG | |
| | NVFSCSVMHEALHNHYTQKSLSLSPGK | |
| First polypeptide | QVQLVESGGGVVQPGRSLRLDCKASGITFSNSGMHWVRQAPGKGL | 19 |
| chain of | EWVAVIWYDGSKRYYADSVKGRFTISRDNSKNTLFLQMNSLRAEDT | |
| immunoconjugate | AVYYCATNDDYWGQGTLVTVSSASTKGPSVFPLAPSSKSTSGGTAA | |
| 2-12 | LGCLVKDYFPEPVTVSWNSGALTSGVHTFPAVLQSSGLYSLSSVVTV | |
| | PSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSN | |
| | KALGAPIEKTISKAKGQPREPQVYTLPPCRDELTKNQVSLWCLVKGF | |
| | YPSDIAVEWESNGQPENNYKTTPPVLDSDGSFFLYSKLTVDKSRWQQ | |
| | GNVFSCSVMHEALHNHYTQKSLSLSPGGGGGSAPASSSTKKTQLQL | |
| | EHLLLDLQMILNGINNYKNPKLTLMLTAKFYMPKKATELKHLQCLE | |
| | EELKPLEEVLNLAQSKNFHLRPRDLISNINVIVLELKGSETTFMCEYA | |
| | DETATIVEFLNRWITFAQSIISTLT | |
| Second polypeptide | QVQLVESGGGVVQPGRSLRLDCKASGITFSNSGMHWVRQAPGKGL | 20 |
| chain of | EWVAVIWYDGSKRYYADSVKGRFTISRDNSKNTLFLQMNSLRAEDT | |
| immunoconjugate | AVYYCATNDDYWGQGTLVTVSSASTKGPSVFPLAPSSKSTSGGTAA | |
| 2-12 | LGCLVKDYFPEPVTVSWNSGALTSGVHTFPAVLQSSGLYSLSSVVTV | |
| | PSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSN | |
| | KALGAPIEKTISKAKGQPREPQVCTLPPSRDELTKNQVSLSCAVKGF | |
| | YPSDIAVEWESNGQPENNYKTTPPVLDSDGSFFLVSKLTVDKSRWQQ | |
| | GNVFSCSVMHEALHNHYTQKSLSLSPGK | |
| First polypeptide | QVQLVESGGGVVQPGRSLRLDCKASGITFSNSGMHWVRQAPGKGL | 21 |
| chain of | EWVAVIWYDGSKRYYADSVKGRFTISRDNSKNTLFLQMNSLRAEDT | |
| immunoconjugate | AVYYCATNDDYWGQGTLVTVSSASTKGPSVFPLAPSSKSTSGGTAA | |
| 2-15 | LGCLVKDYFPEPVTVSWNSGALTSGVHTFPAVLQSSGLYSLSSVVTV | |
| | PSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSN | |
| | KALGAPIEKTISKAKGQPREPQVYTLPPCRDELTKNQVSLWCLVKGF | |
| | YPSDIAVEWESNGQPENNYKTTPPVLDSDGSFFLYSKLTVDKSRWQQ | |
| | GNVFSCSVMHEALHNHYTQKSLSLSPGGGGGSAPASSSTKKTQLQL | |
| | EHLLLDLQMILNGINNYKNPKLTLMLTAKFYMPKKATELKHLQCLE | |
| | EELKPLEEVLNLAQSKNFHLRPRDLISDINVIVLELKGSETTFMCEYA | |
| | DETATIVEFLNRWITFAQSIISTLT | |
| Second polypeptide | QVQLVESGGGVVQPGRSLRLDCKASGITFSNSGMHWVRQAPGKGL | 22 |
| chain of | EWVAVIWYDGSKRYYADSVKGRFTISRDNSKNTLFLQMNSLRAEDT | |
| immunoconjugate | AVYYCATNDDYWGQGTLVTVSSASTKGPSVFPLAPSSKSTSGGTAA | |
| 2-15 | LGCLVKDYFPEPVTVSWNSGALTSGVHTFPAVLQSSGLYSLSSVVTV | |
| | PSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSN | |
| | KALGAPIEKTISKAKGQPREPQVCTLPPSRDELTKNQVSLSCAVKGF | |
| | YPSDIAVEWESNGQPENNYKTTPPVLDSDGSFFLVSKLTVDKSRWQQ | |
| | GNVFSCSVMHEALHNHYTQKSLSLSPGK | |
| First polypeptide | APASSSTKKTQLQLEHLLLDLQMILNGINNYKNPKLTLMLTAKFYMP | 23 |
| chain of | KKATELKHLQCLEEELKPLEEVLNLAQSKNFHLRPRDLISDINVIVLE | |
| immunoconjugate | LKGSETTFMCEYADETATIVEFLNRWITFSQSIISTLTGGGGSDKTHTC | |
| 2-16 | PPCPAPEAAGGPSVFLFPPKPKDTLMISRTPEVTCVVVDVSHEDPEV | |
| | KFNWYVDGVEVHNAKTKPREEQYNSTYRVVSVLTVLHQDWLNGK | |
| | EYKCKVSNKALPAPIEKTISKAKGQPREPQVYTLPPCRDELTKNQVS | |
| | LWCLVKGFYPSDIAVEWESNGQPENNYKTTPPVLDSDGSFFLYSKLT | |
| | VDKSRWQQGNVFSCSVMHEALHNHYTQKSLSLSPGK | |
| Second polypeptide | QVQLVESGGGVVQPGRSLRLDCKASGITFSNSGMHWVRQAPGKGL | 24 |
| chain of | EWVAVIWYDGSKRYYADSVKGRFTISRDNSKNTLFLQMNSLRAEDT | |
| immunoconjugate | AVYYCATNDDYWGQGTLVTVSSASTKGPSVFPLAPSSKSTSGGTAA | |
| 2-16 | LGCLVKDYFPEPVTVSWNSGALTSGVHTFPAVLQSSGLYSLSSVVTV | |
| | PSSSLGTQTYICNVNHKPSNTKVDKKVEPKSCDKTHTCPPCPAPEAA | |
| | GGPSVFLFPPKPKDTLMISRTPEVTCVVVDVSHEDPEVKFNWYVDG | |
| | VEVHNAKTKPREEQYNSTYRVVSVLTVLHQDWLNGKEYKCKVSN | |
| | KALPAPIEKTISKAKGQPREPQVCTLPPSRDELTKNQVSLSCAVKGFY | |
| | PSDIAVEWESNGQPENNYKTTPPVLDSDGSFFLVSKLTVDKSRWQQG | |
| | NVFSCSVMHEALHNHYTQKSLSLSPGK | |
| First polypeptide | EVQLLESGGGLVQPGGSLRLSCAASGFSFSSYTMSWVRQAPGKGLE | 25 |
| chain of | WVATISGGGRDIYYPDSVKGRFTISRDNSKNTLYLQMNSLRAEDTAV | |
| immunoconjugate | YYCVLLTGRVYFALDSWGQGTLVTVSSASTKGPSVFPLAPSSKSTSG | |
| 3-1 | GTAALGCLVKDYFPEPVTVSWNSGALTSGVHTFPAVLQSSGLYSLSS | |
| | VVTVPSSSLGTQTYICNVNHKPSNTKVDKKVEPKSCDKTHTCPPCPA | |
| | PEAAGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALGAPIEKTISKAKGQPREPQVYTLPPCRDELTKNQVSLWCL | |
| | VKGFYPSDIAVEWESNGQPENNYKTTPPVLDSDGSFFLYSKLTVDKS | |
| | RWQQGNVFSCSVMHEALHNHYTQKSLSLSPGGGGGSGGGGSGGG | |
| | GSAPASSSTKKTQLQLEHLLLDLQMILNGINNYKNPKLTRMLTAKFA | |
| | MPKKATELKHLQCLEEELKPLEEVLNGAQSKNFHLRPRDLISNINVI | |
| | VLELKGSETTFMCEYADETATIVEFLNRWITFAQSIISTLT | |
| Second polypeptide | EVQLLESGGGLVQPGGSLRLSCAASGFSFSSYTMSWVRQAPGKGLE | 26 |
| chain of | WVATISGGGRDIYYPDSVKGRFTISRDNSKNTLYLQMNSLRAEDTAV | |
| immunoconjugate | YYCVLLTGRVYFALDSWGQGTLVTVSSASTKGPSVFPLAPSSKSTSG | |
| 3-1 | GTAALGCLVKDYFPEPVTVSWNSGALTSGVHTFPAVLQSSGLYSLSS | |
| | VVTVPSSSLGTQTYICNVNHKPSNTKVDKKVEPKSCDKTHTCPPCPA | |
| | PEAAGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALGAPIEKTISKAKGQPREPQVCTLPPSRDELTKNQVSLSCA | |
| | VKGFYPSDIAVEWESNGQPENNYKTTPPVLDSDGSFFLVSKLTVDKS | |
| | RWQQGNVFSCSVMHEALHNHYTQKSLSLSPGK | |
| First polypeptide | APASSSTKKTQLQLEHLLLDLQMILNGINNYKNPKLTRMLTFKFYMP | 27 |
| chain of | KKATELKHLQCLEEELKPLEEVLNLAGDASIHDLISRINVIVLELKGS | |
| immunoconjugate | ETTFMCEYADETATIVEFLNRWITFSQSIIRTLTGGGGSGGGGSDKTH | |
| 3-4 | TCPPCPAPEAAGGPSVFLFPPKPKDTLMISRTPEVTCVVVDVSHEDPE | |
| | VKFNWYVDGVEVHNAKTKPREEQYNSTYRVVSVLTVLHQDWLNG | |
| | KEYKCKVSNKALPAPIEKTISKAKGQPREPQVYTLPPCRDELTKNQV | |
| | SLWCLVKGFYPSDIAVEWESNGQPENNYKTTPPVLDSDGSFFLYSKL | |
| | TVDKSRWQQGNVFSCSVMHEALHNHYTQKSLSLSPGK | |
| Second polypeptide | QVQLVQSGAEVKKPGSSVKVSCKASGGTFSSYAISWVRQAPGQGLE | 28 |
| chain of | WMGLIIPMFDTAGYAQKFQGRVAITVDESTSTAYMELSSLRSEDTAV | |
| immunoconjugate | YYCARAEHSSTGTFDYWGQGTLVTVSSASTKGPSVFPLAPSSKSTSG | |
| 3-4 | GTAALGCLVKDYFPEPVTVSWNSGALTSGVHTFPAVLQSSGLYSLSS | |
| | VVTVPSSSLGTQTYICNVNHKPSNTKVDKKVEPKSCDKTHTCPPCPA | |
| | PEAAGGPSVFLFPPKPKDTLMISRTPEVTCVVVDVSHEDPEVKFNW | |
| | YVDGVEVHNAKTKPREEQYNSTYRVVSVLTVLHQDWLNGKEYKC | |
| | KVSNKALPAPIEKTISKAKGQPREPQVCTLPPSRDELTKNQVSLSCAV | |
| | KGFYPSDIAVEWESNGQPENNYKTTPPVLDSDGSFFLVSKLTVDKSR | |
| | WQQGNVFSCSVMHEALHNHYTQKSLSLSPGK | |
| Nivolumab light | RASQSVSSYLA | 29 |
| chain LCDR1 | | |
| Nivolumab light | DASNRAT | 30 |
| chain LCDR2 | | |
| Nivolumab light | QQSSNWPRT | 31 |
| chain LCDR3 | | |
| Nivolumab heavy | NSGMH | 32 |
| chain HCDR1 | | |
| Nivolumab heavy | VIWYDGSKRYYADSVKG | 33 |
| chain HCDR2 | | |
| Nivolumab heavy | NDDY | 34 |
| chain HCDR3 | | |
| Nivolumab light | EIVLTQSPATLSLSPGERATLSCRASQSVSSYLAWYQQKPGQAPRLLI | 35 |
| chain variable | YDASNRATGIPARFSGSGSGTDFTLTISSLEPEDFAVYYCQQSSNWPR | |
| region VL | TFGQGTKVEIK | |
| Nivolumab heavy | QVQLVESGGGVVQPGRSLRLDCKASGITFSNSGMHWVRQAPGKGL | 36 |
| chain variable | EWVAVIWYDGSKRYYADSVKGRFTISRDNSKNTLFLQMNSLRAEDT | |
| region VH | AVYYCATNDDYWGQGTLVTVSS | |

1. An immunoconjugate, comprising a first polypeptide chain comprising, from N-terminus to C-terminus, a heavy chain Fd fragment of an anti-PD-1 antibody, an Fc, a linker, and a laIL-2 in sequence, wherein laIL-2 is linked to the C-terminus of the Fc via the linker; a second polypeptide chain comprising a heavy chain Fd fragment of the anti-PD-1 antibody and an Fc; and two third polypeptide chains each comprising one light chain of the anti-PD-1 antibody,
wherein laIL-2, as compared to wild-type IL-2, comprises amino acid mutations R38L, F42A, and N88D, wherein the amino acid sequence of the wild-type IL-2 is the amino acid sequence set forth in SEQ ID NO: 1; and
wherein the anti-PD-1 antibody comprises: an LCDR1 of the amino acid sequence set forth in SEQ ID NO: 29, an LCDR2 of the amino acid sequence set forth in SEQ ID NO: 30, an LCDR3 of the amino acid sequence set forth in SEQ ID NO: 31, an HCDR1 of the amino acid sequence set forth in SEQ ID NO: 32, an HCDR2 of the amino acid sequence set forth in SEQ ID NO: 33, and an HCDR3 of the amino acid sequence set forth in SEQ ID NO: 34.

2. The immunoconjugate according to claim 1, wherein laIL-2, as compared to a wild-type IL-2, further comprises a mutation at position 3 and/or position 125, wherein the mutation at position 3 is T3A, the mutation at position 125 is C125A or C125S.

3. The immunoconjugate according to claim 1, wherein laIL-2 comprises an amino acid sequence set forth in SEQ ID NO: 3 or 5, or an amino acid sequence having at least 90%, 91%, 92%, 93%, 94%, 95%, 96%, 97%, 98%, or 99% identity thereto.

4.-5. (canceled)

6. The immunoconjugate according to claim 1, wherein the anti-PD-1 antibody comprises:
a light chain variable region comprising the amino acid sequence set forth in SEQ ID NO: 35; and a heavy chain variable region comprising the amino acid sequence set forth in SEQ ID NO: 36.

7. The immunoconjugate according to claim 1, wherein the Fc is derived from IgG1, IgG2, IgG3, or IgG4; or, the Fc is derived from IgG1.

8. (canceled)

9. The immunoconjugate according to claim 1, wherein one polypeptide chain of the Fc comprises T366W and S354C mutations, and the other polypeptide chain comprises T366S, L368A, Y407V, and Y349C mutations; or, the Fc further comprises an LALA mutation or an LALAPG mutation.

10. The immunoconjugate according to claim 1, wherein the linker is 1G4S, 2G4S, or 3G4S; or the linker is 1G4S.

11. The immunoconjugate according to claim 1, wherein the Fc comprises an amino acid sequence selected from the group consisting of amino acid sequences set forth in SEQ ID NOs: 9-12.

12. The immunoconjugate according to claim 1, wherein the heavy chain Fd fragment of the anti-PD-1 antibody comprises the amino acid sequence set forth in SEQ ID NO: 14.

13. The immunoconjugate according to claim 8, wherein the light chain of the anti-PD-1 antibody comprises the amino acid sequence set forth in SEQ ID NO: 13.

14. The immunoconjugate according to claim 1, wherein the immunoconjugate comprises:
a first polypeptide chain comprising, from N-terminus to C-terminus, a heavy chain Fd fragment of the anti-PD-1 antibody, an Fc, 1G4S, and laIL-2 in sequence, wherein laIL-2 is linked to the C-terminus of the Fc via 1G4S, and amino acid sequences of the heavy chain Fd fragment, the Fc, and laIL-2 are amino acid sequences set forth in SEQ ID NOs: 14, 11, and 3, respectively; a second polypeptide chain comprising a heavy chain Fd fragment of the anti-PD-1 antibody and an Fc, wherein amino acid sequences of the heavy chain Fd fragment and the Fc are amino acid sequences set forth in SEQ ID NOs: 14 and 12, respectively; and two third polypeptide chains each comprising one light chain of the anti-PD-1 antibody, wherein an amino acid sequence of the light chain is the amino acid sequence set forth in SEQ ID NO: 13.

15. The immunoconjugate according to claim 1, comprising:
a first polypeptide chain comprising the amino acid sequence set forth in SEQ ID NO: 21;
a second polypeptide chain comprising the amino acid sequence set forth in SEQ ID NO: 22; and
two third polypeptide chains each comprising the amino acid sequence set forth in SEQ ID NO: 13.

16. A nucleic acid encoding the immunoconjugate according to claim 1.

17. A vector comprising the nucleic acid according to claim 16.

18. A cell comprising the nucleic acid according to claim 16 or a vector, wherein the vector comprises the nucleic acid.

19. A pharmaceutical composition comprising the immunoconjugate according to claim 1, a nucleic acid, a vector, and/or a cell;
wherein the nucleic acid encodes the immunoconjugate, the vector comprises the nucleic acid, and the cell comprises the nucleic acid or the vector;
or, the pharmaceutical composition further comprises an additional therapeutic agent comprising a small-molecule drug, a nucleic acid drug, an antibody or an antibody fragment, and/or a polypeptide for use in treating cancer;
or, the pharmaceutical composition further comprises an excipient.

20. A method for preventing and/or treating cancer in a subject in need thereof, comprising administering to the subject a therapeutically effective amount of the immunoconjugate according to claim 1, a nucleic acid, a vector, a cell, or a pharmaceutical composition;
wherein the nucleic acid encodes the immunoconjugate, the vector comprises the nucleic acid, the cell comprises the nucleic acid or the vector, and the pharmaceutical composition comprises the immunoconjugate, the nucleic acid, the vector and/or the cell;
or, the cancer is a solid tumor, a hematologic tumor, or an anti-PD-1/PD-L1 antibody treatment-resistant cancer; the solid tumor is non-small cell lung cancer, small cell lung cancer, esophageal cancer, gastric cancer, gastroesophageal junction cancer, colorectal cancer, melanoma, malignant pleural mesothelioma, cervical cancer, biliary tract cancer, cholangiocarcinoma, endometrial cancer, cancer with a high tumor mutational burden, cutaneous squamous cell carcinoma, hepatocellular carcinoma, pancreatic ductal adenocarcinoma, renal cell carcinoma, urothelial carcinoma, prostate cancer, ovarian cancer, triple-negative breast cancer, head and neck squamous cell carcinoma, nasopharyngeal carcinoma, glioblastoma, anaplastic thyroid carcinoma, Merkel cell carcinoma, or soft tissue sarcoma; the hematologic tumor is Hodgkin lymphoma, lymphoma, primary mediastinal large B-cell lymphoma, leukemia, multiple myeloma, or myelofibrosis.


SEQUENCE LISTING (36 sequences)

SEQ ID NO: 1 - protein, 133 aa, Homo sapiens [sequence omitted]

SEQ ID NO: 2 - protein, 133 aa, synthetic construct
APASSSTKKTQLQLEHLLLDLQMILNGINNYKNPKLTLMLTAKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 3 - protein, 133 aa, synthetic construct
APASSSTKKTQLQLEHLLLDLQMILNGINNYKNPKLTLMLTAKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 4 - protein, 133 aa, synthetic construct
APASSSTKKTQLQLEHLLLDLQMILNGINNYKNPKLTLMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 5 - protein, 133 aa, synthetic construct
APASSSTKKTQLQLEHLLLDLQMILNGINNYKNPKLTLMLTAKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 6 - protein, 5 aa, synthetic construct
GGGGS

SEQ ID NO: 7 - protein, 10 aa, synthetic construct
GGGGSGGGGS

SEQ ID NO: 8 - protein, 15 aa, synthetic construct
GGGGSGGGGSGGGGS

SEQ ID NO: 9 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 216 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 217 aa, synthetic construct
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 13 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 211 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 15 - protein, 218 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 214 aa, synthetic construct
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 365 aa, synthetic construct
APASSSTKKTQLQLEHLLLDLQMILNGINNYKNPKLTLMLTAKFYMPKKATELKHLQCLEEELKPLEEVLNLAQSKNFHLRPRDLISNINVIVLELKGSETTFMCEYADETATIVEFLNRWITFSQSIISTLTGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 443 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 19 - protein, 580 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSAPASSSTKKTQLQLEHLLLDLQMILNGINNYKNPKLTLMLTAKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 20 - protein, 443 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 580 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSAPASSSTKKTQLQLEHLLLDLQMILNGINNYKNPKLTLMLTAKFYMPKKATELKHLQCLEEELKPLEEVLNLAQSKNFHLRPRDLISDINVIVLELKGSETTFMCEYADETATIVEFLNRWITFAQSIISTLT

SEQ ID NO: 22 - protein, 443 aa, synthetic construct; identical to SEQ ID NO: 20

SEQ ID NO: 23 - protein, 365 aa, synthetic construct
APASSSTKKTQLQLEHLLLDLQMILNGINNYKNPKLTLMLTAKFYMPKKATELKHLQCLEEELKPLEEVLNLAQSKNFHLRPRDLISDINVIVLELKGSETTFMCEYADETATIVEFLNRWITFSQSIISTLTGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 443 aa, synthetic construct; identical to SEQ ID NO: 18

SEQ ID NO: 25 - protein, 597 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 26 - protein, 450 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 27 - protein, 366 aa, synthetic construct
APASSSTKKTQLQLEHLLLDLQMILNGINNYKNPKLTRMLTFKFYMPKKATELKHLQCLEEELKPLEEVLNLAGDASIHDLISRINVIVLELKGSETTFMCEYADETATIVEFLNRWITFSQSIIRTLTGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 450 aa, synthetic construct
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 11 aa, Homo sapiens
RASQSVSSYLA

SEQ ID NO: 30 - protein, 7 aa, Homo sapiens
DASNRAT

SEQ ID NO: 31 - protein, 9 aa, Homo sapiens
QQSSNWPRT

SEQ ID NO: 32 - protein, 5 aa, Homo sapiens
NSGMH

SEQ ID NO: 33 - protein, 17 aa, Homo sapiens
VIWYDGSKRYYADSVKG

SEQ ID NO: 34 - protein, 4 aa, Homo sapiens
NDDY

SEQ ID NO: 35 - protein, 107 aa, Homo sapiens [sequence omitted]

SEQ ID NO: 36 - protein, 113 aa, Homo sapiens [sequence omitted]